(19) United States
(12) Patent Application Publication
(10) Pub. No.: US 2026/0131079 A1
(43) Pub. Date: May 14, 2026

(54) Lossy Mechatronic Systems And Methods Of Estimation

(71) Applicant: Cepheid, Sunnyvale, CA (US)

(72) Inventors: Rajesh Nerkar, Sunnyvale, CA (US); Amrita CHIMNI, Sunnyvale, CA (US); Yajing LIU, Sunnyvale, CA (US); Janaki-Rama VEGIRAJU, Sunnyvale, CA (US); Richard J. CASLER, JR., Sunnyvale, CA (US)

(73) Assignee: Cepheid, Sunnyvale, CA (US)

(21) Appl. No.: 19/389,938

(22) Filed: Nov. 14, 2025

Related U.S. Application Data
(63) Continuation of application No. 17/575,009, filed on Jan. 13, 2022, now abandoned.
(60) Provisional application No. 63/136,739, filed on Jan. 13, 2021.

(51) Int. Cl. A61M 5/172; A61M 5/168

(52) U.S. Cl. CPC A61M 5/172; A61M 5/16831; A61M 2205/332; A61M 2205/50

(57) ABSTRACT

Methods and systems for estimating force and motor torque in a mechatronic system are provided herein. Such methods and system are suited for improved control of small-scale mechatronic system, particularly a syringe, valve, and cartridge loader or door opening/closing mechanism of a diagnostic assay system. The methods can compensate for friction and account for various second-order effects, thereby allowing for more accurate pressure estimation, thereby allowing improved syringe operation. The methods can further allow for improved estimation of force or motor torque to allow for improved control of an actuatable valve interfacing the sample cartridge and cartridge loader or door opening/closing system. Methods of calibrating such systems are also provided.

### CROSS-REFERENCES TO RELATED APPLICATIONS

This application is a continuation application of U.S. Non-Provisional application Ser. No. 17/575,009, filed Jan. 13, 2022, which claims the benefit of priority of U.S. Provisional Application No. 63/136,739 filed on Jan. 13, 2021, the entire contents of which are incorporated herein by reference.

### STATEMENT AS TO RIGHTS TO INVENTIONS MADE UNDER FEDERALLY SPONSORED RESEARCH AND DEVELOPMENT

This invention was made with U.S. Government support under Agreement No. W15QKN-16-9-1002 awarded by the ACC-NJ to the MCDC. The Government has certain rights in the invention.

### BACKGROUND OF THE INVENTION

The present invention pertains to improved device and methods for control of mechatronic systems, particularly small-scale, high precision devices in the diagnostics field.

This application is generally related to: U.S. Pat. No. 10,562,030 entitled “Molecular Diagnostic Assay System,” issued Feb. 18, 2020; U.S. Pat. No. 10,348,225 entitled “Encoderless Motor with Improved Granularity and Methods of Use” issued Jul. 9, 2019; U.S. Provisional Patent Application No. 63/136,766 entitled “Encoderless Motor with Improved Quantization and Methods of Use and Calibration” filed Jan. 13, 2021; U.S. Pat. No. 8,048,386 entitled “Fluid Processing and Control,” filed Feb. 25, 2002; and U.S. Pat. No. 6,374,684 entitled “Fluid Control and Processing System,” filed Aug. 25, 2000; each of which is incorporated herein by reference in its entirety for all purposes.

The present inventors have developed methods and systems that improve upon existing molecular diagnostic assay systems (e.g., Cepheid's GeneXpert® system). The new molecular diagnostic assay systems and methods described herein pertain to a medical diagnostic device, which is optionally powered by battery, typically small in size and light in weight, thus permitting complete portable use at any location where patients may be, away from hospitals, laboratories, or even drug stores. The diagnostic device is capable of performing fully automated molecular diagnostic assays (optionally for detecting multiple pathogens at the same time), rapidly obtain accurate results (typically within 1 or 2 hours and as fast as 15-20 minutes). It is easy to operate, using one or more pre-manufactured assay cartridges one can quickly obtain test results indicating whether a patient is carrying particular pathogen(s), or afflicted with a particular disease state.

These molecular diagnostic assay systems are the first true point-of-care diagnostic tool possessing the strength of rapid deployment and full operation in virtually any environment. The combination of its deploy ability, its rapid and accurate diagnostic functionality, its technical sophistication yet ease of operation, makes these molecular diagnostic assay systems the ultimate solution for the emerging markets and the revolutionary trend-setter that defines the future of medical diagnostic testing. While the development of these portable diagnostic systems represents marked advancements in the state of the art in recent years, there is a need for continued improvement in accuracy of control of such systems, particularly for small-scale mechatronic systems disposed within, to ensure reliability and consistency of results.

### BRIEF SUMMARY OF THE INVENTION

The invention relates to improved diagnostic assay systems and methods of control and estimation. Such systems can include improvements pertaining to various subassemblies including: a door drive assembly, a cartridge loader, a syringe drive and a valve drive. It is appreciated that any of these subassemblies can be included in such a diagnostic assay system separately or in combination with any other subassembly to provide improved performance aspects as described herein.

In one aspect, the invention pertains to a lossy, mechatronic system for controlling at least one of a position, a velocity or a generalized force. Here the term generalized force shall be taken to mean a force, torque or pressure output of the mechatronic system. In some embodiments, the system includes: a motor driver; a motor configured to apply a generalized force in accordance with the motor driver; a lossy transmission configured to deliver a generalized force in accordance with the motor-applied generalized force and a friction and a viscous drag, and a control unit. The control unit can include a processor with a memory having instructions recorded thereon for computing the generalized forces using at least one motor characteristic, a motor drive bridge current and voltage and at least one transmission characteristic. Advantageously, the control unit can perform these computations in real-time. The motor characteristic can include any of: a voltage, a velocity, a position, a phase current, a phase resistance, and a motor constant (kt). The transmission characteristic comprises any of: a transmission gear ratio, coefficient-of-friction, and a viscous drag coefficient. Any of the embodiments herein can be applied in at least one of a syringe drive, a valve drive, a cartridge loader, or door opening/closing mechanism

In some embodiments, the transmission is backdrivable. The transmission can be enabled for four-quadrant operation. In one aspect, the backdrivable transmission allows forces applied by the user to be used as inputs. In some embodiments, users of the system can impart generalized forces on the output and sense the generalized force at the input and thereby communicate user intent. Examples could include but not limited to the user pushing upward on the door or syringe to signal intent to clean the instrument or syringe rod. In some embodiments, the system includes a cartridge loading system in which the user action of pushing on the cartridge against the cartridge loading cam mechanism signals a user request to load the cartridge and start processing the cartridge. In some embodiments, the transmission is a rotary transmission with output torque representing the generalized force output. In some embodiments, the transmission is a linear transmission with output force representing the generalized force output—a force or a pressure for instance.

In some embodiments, the control unit is configured to: determine motor resistance by a motor drive voltage, a motor drive bridge current and a motor drive bridge voltage. In one aspect, the motor windings are of known conductor composition and the motor resistance is further determined at a known winding temperature. These known values can be stored in the non-volatile memory of the control unit. In some embodiments, the control unit determines the motor winding temperature from the known relationship between motor winding resistance and the winding temperature, which can be determined in real-time. In some embodiments, the motor windings are constructed with substantially copper composition. The motor winding temperature can be used to compensate for the impact of winding temperature on the generalized force output. In some embodiments, the system operation is shut down when the motor winding temperature exceeds a pre-determined threshold.

In some embodiments, the system includes a syringe drive and the generalized force output is used in a guarded, stop-on-force motion of the syringe during one or more operations. These operations can include any of: locating cartridge bottom with the syringe, detecting excessive aspirating and/or dispensing force while performing at least one of mixing or reaction-tube filling with the syringe, and determining a sample-volume adequacy. In some embodiments, the guarded, stop-on-force motion is a stop-on-pressure. In some embodiments, the system can be applied as a syringe drive and the generalized force output is used as a means of determining cartridge integrity. In some embodiments, the cartridge integrity is determined by sensing a loss of pressurization due to a leak in the reaction-vessel within a cartridge integrity test. In some embodiments the guarded, stop-on-force motion is employed as a risk control measure to sense obstruction, like the finger of a user obstructing door closure.

In another aspect a calibration method for application of a lossy mechatronic system, such as any of those described above, is provided herein. The calibration method can include: determining a motor winding resistance, and extending a transmission and then retracting the transmission while driving into a compliant, instrumented platform; recording a reading from the instrumented platform and a generalized force; and computing, by processing the recordings by the platform to determine a motor kt and a coefficient-of-friction. In some embodiments, the motor kt and the coefficient-of-friction are stored on a memory of a control unit of the lossy mechatronic system to facilitate accurate operation of the lossy mechatronic system within +/−10% accuracy. In some embodiments, the transmission is backdrivable enabling four-quadrant operation. In some embodiments, the system output is linear. In some embodiments, the linear output system is a syringe drive.

In some embodiments, the invention includes a diagnostic assay system adapted to receive an assay cartridge (also referred to occasionally as a “sample cartridge” or “test cartridge”). Such systems can include any one or combination of the various features and subassemblies described herein.

In some embodiments, the system includes a brushless DC (BLDC) motor operatively coupled with, for example, any of a door opening/closing mechanism and cartridge loading system, a syringe drive, and/or a valve drive.

In some embodiments, the system includes a door opening/closing mechanism. In some embodiments, the system includes a cartridge loading mechanism. In some embodiments, the system includes a door opening/closing mechanisms cooperatively coupled with a cartridge loading mechanism and driven by a backdrivable transmission mechanism.

In some embodiments, the system includes a syringe drive operatively coupled with a n-phase BLDC motor and controlled based at least in-part on monitored current draw of the BLDC motor.

In some embodiments, the system includes at least one of a syringe drive, a cartridge loading mechanism, a door mechanism and a valve drive mechanism operatively coupled with a n-phase BLDC motor based at least in-part on a voltage signal provided by n voltage sensors of the BLDC—each sensing the magnetic field of the rotor poles-without use of any extrinsic encoder hardware or position sensors.

Some embodiments of the invention relate to a door operating system for a diagnostic assay system. The system can include a chassis of the diagnostic assay system. A brushless DC (BLDC) motor can be coupled to the chassis of the diagnostic assay system. A backdrivable transmission can be operable by the BLDC motor. A door can be movable relative to the chassis of the diagnostic assay system from a closed position to an open position (and from an open position to a closed position). The BLDC motor can be configured to operate the backdrivable transmission based on current measurements of the BLDC motor, the current measurements being associated with backdriving events against the backdrivable transmission. Here, the term backdrivable shall be taken in the classical robotic context as the level of easiness of the transmission of movement from the output of the transmission to the motor drive input to the transmission.

Some embodiments of the invention relate to a method for operating a door opening/closing system for a diagnostic assay system. In the method, a command can be received to open a cartridge receiving door of the diagnostic assay system. A brushless DC (BLDC) motor coupled to a backdrivable transmission can be operated to open the door from a closed position (and vice versa), the backdrivable transmission being operationally coupled to the door and a cartridge loading mechanism. A first backdriving event, say a user pushing up on the door, occurring against the backdrivable transmission can be detected, based on monitoring of the current. Based on detecting the first backdriving event, operation of the BLDC motor to place the door in an open position can be ceased, and an aspect of the cartridge loading mechanism can be placed into position for accepting an assay cartridge.

Some embodiments of the invention relate to a system for operating a syringe for a diagnostic assay system. The system can include a chassis of a diagnostic assay system. A brushless DC (BLDC) motor can be coupled to the chassis of the diagnostic assay system. A backdrivable lead screw can be operable by the BLDC motor. A plunger rod can be operable by the lead screw to engage a plunger tip in a syringe passage of the assay cartridge. The BLDC motor can be configured to operate the lead screw based on monitoring current consumption of the BLDC motor, the current being associated with pressure changes within the removable assay cartridge.

Some embodiments of the invention relate to a method for operating a syringe for a diagnostic assay system. A command to power a brushless DC (BLDC) motor can be received. The BLDC motor can be operable to turn a backdrivable lead screw. A plunger rod can be coupled to and movable by the lead screw. Power to the BLDC motor can be applied to move the plunger rod to engage a plunger tip within a syringe passage of an assay cartridge. At least one current associated with operation of the BLDC motor can be monitored to determine a quality of the removable assay cartridge. A change in the current of the BLDC motor can be detected. Operation of the BLDC motor can be altered within the removable assay cartridge based on detecting the change in the current.

Some embodiments of the invention relate to a method for operating a valve drive mechanism. A command can be received to power a brushless DC (BLDC) motor coupled to the chassis to move a valve drive to a particular position. The valve drive can be configured to rotate positions of a valve body of a removable assay cartridge. A transmission can be coupled between the BLDC motor and the valve drive. The BLDC motor does not include any extrinsic positional sensors or encoder hardware, but can include a plurality of Hall-effect sensors that measure the rotor magnetic field. The BLDC motor can be powered to rotate a shaft of the BLDC motor a particular number of turns to move the valve drive to the particular position based on a sinusoidal signal generated by the sensors.

Some embodiments relate to a system for operating a valve drive mechanism. The system can include a valve drive mechanism chassis. A brushless DC (BLDC) motor can be coupled to the chassis. The BLDC motor does not include any extrinsic positional or encoder hardware but can include a plurality of Hall-effect sensors. A transmission can be coupled to BLDC motor. A valve drive can be coupled to the transmission. The valve drive can be configured to rotate positions of a valve body of a removable assay cartridge. Position of the valve drive output can be determined based on analyzing signals generated by the sensors.

### BRIEF DESCRIPTION OF THE DRAWINGS

FIG. 1A is a perspective view of a diagnostic assay system, in accordance with some embodiments of the invention.

FIG. 1B is an exploded view of a diagnostic assay system, in accordance with some embodiments.

FIGS. 2A-2C are perspective views of a brushless DC (BLDC) motor, in accordance with some embodiments.

FIG. 2D is a graph of a sinusoidal variable voltage output pattern of intrinsic magnetic field sensors in proximity to a BLDC motor used to determine the mechanical angular position of the rotor of the motor, in accordance with some embodiments.

FIG. 3 is a circuit diagram for controlling a BLDC motor, in accordance with some embodiments.

FIG. 4A is a perspective view of a door opening mechanism, in accordance with some embodiments.

FIGS. 4B-4E are cross sectional views of a diagnostic assay system in use, in accordance with some embodiments.

FIG. 5A is a cross sectional view of a diagnostic assay system in use, in accordance with some embodiments.

FIGS. 5B and 5C are flow diagrams of a method for operating aspects of a diagnostic assay system, in accordance with some embodiments.

FIGS. 6A and 6B are perspective views of a valve drive mechanism, in accordance with some embodiments.

FIG. 6C is a graph relating an output signal to valve drive position, in accordance with some embodiments.

FIGS. 7-8 illustrates an ultrasonic horn assembly for use in diagnostic assay system, in accordance with some embodiments.

FIGS. 9A-B illustrates cross-sectional views of a diagnostic assay system during and after loading of an assay cartridge, in accordance with some embodiments.

FIG. 10 illustrates cross-sectional view of an assay cartridge in accordance with some embodiments of the invention.

FIGS. 11-12 illustrates pressure sensing control diagrams, in accordance with some embodiments.

FIG. 13 illustrates modeling of a leadscrew actuator transmission as a transformer and

FIG. 14 illustrates a corresponding control diagram, in accordance with some embodiments.

FIG. 15 illustrates modeling of a leadscrew actuator transmission accounting for friction, and FIGS. 16-17 depict correspond control diagrams, in accordance with some embodiments.

FIG. 18 illustrates a control diagram for a mechatronic system, in accordance with some embodiments.

FIG. 19 illustrates a control diagram for a mechatronic system, in accordance with some embodiments.

FIG. 20 is a torque versus angle plot for a mechatronic system, in accordance with some embodiments.

FIGS. 21A-D illustrates estimated syringe pressure (PSI) versus measured pressure (PSI), showing the effects of friction.

FIG. 21B-21D depict alternative methods of pressure sensing, in accordance with some embodiments.

FIG. 21C depict a friction compensation method of pressure sensing, in accordance with some embodiments.

FIGS. 22-23 depict a curve fit of force data for the syringe system, and FIG. 23 illustrates estimated pressure versus measured pressure for the syringe system, in accordance with some embodiments.

FIG. 24 depicts transmission characterization at N=40, and FIG. 25 depicts transmission characterization of a representative motor.

FIG. 26 shows force data from the syringe plotted versus the measured force data, in accordance with some embodiments.

FIG. 27 shows a pressure comparison by using friction compensation methods during pressurization and during depressurization, in accordance with some embodiments.

FIG. 28 shows a pressure comparison by using friction compensation methods during pressurization and during depressurization, in accordance with some embodiments.

FIG. 29 shows a conventional cartridge integrity testing.

FIG. 30 shows an improved cartridge integrity testing, in accordance with some embodiments.

FIG. 31 illustrates cartridge integrity testing results, in accordance with some embodiments.

FIG. 32 illustrates the optimum threshold to detect “good” versus “bad” cartridges cartridge integrity testing in accordance with some embodiments.

FIG. 33 shows a plot of valve torque versus voltage for use in motor torque estimation, in accordance with some embodiments.

### DETAILED DESCRIPTION OF THE INVENTION

### I. System Overview

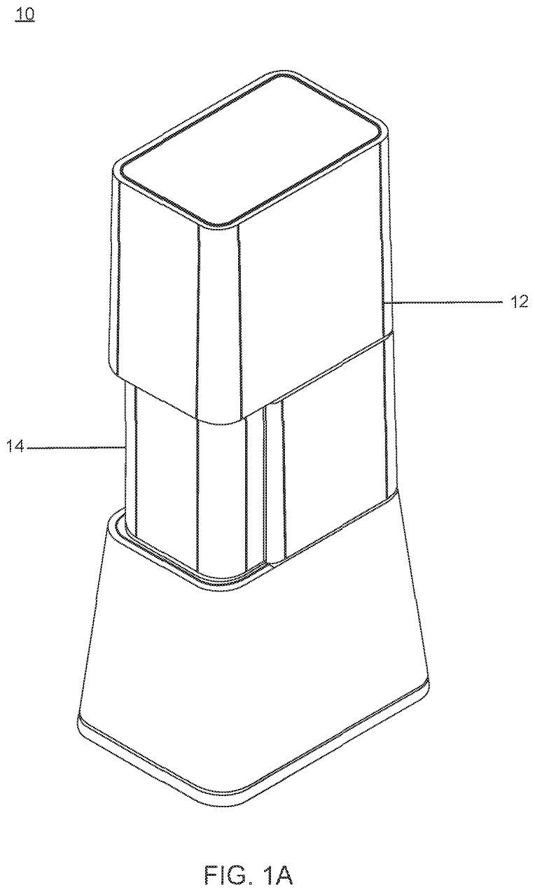

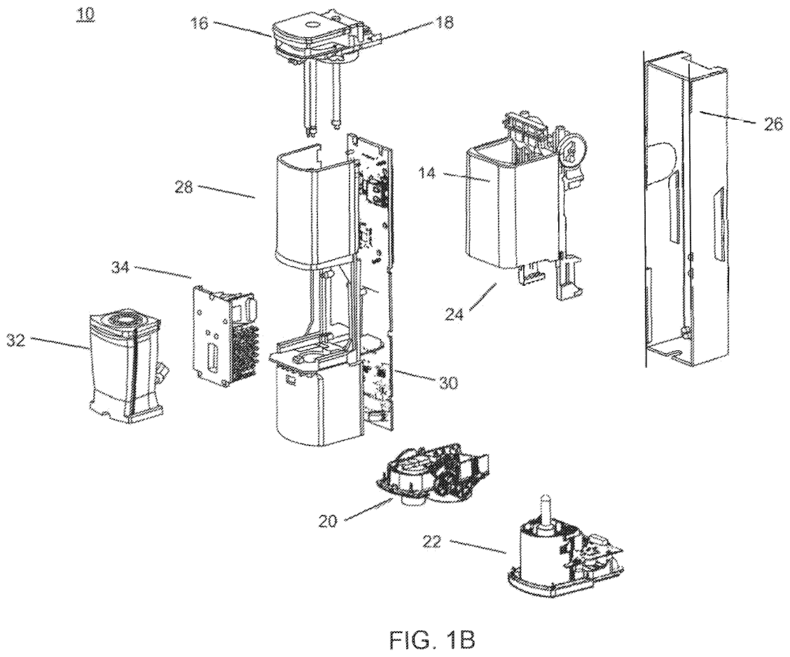

FIG. 1A shows a perspective view of a system 10 for testing a biological sample, according to embodiments of the invention. The compact form factor of the system 10 provides a portable sample testing device that can communicate wirelessly or directly (wired) with a local computer or cloud-based network. As such, the system 10 can be advantageously used for point-of-care applications including mobile diagnostic centers, in emerging countries, and in physician office labs.

The system 10 is usable with a disposable assay cartridge, which is configured to accept a biological sample and adapted for performing a particular assay. The system and cartridges are highly flexible and can be used to detect a variety of analytes, including nucleic acid and protein. Non-limiting exemplary analytes, organisms and disease states that can be detected using the system and assay cartridges includes, nucleic acids, DNA, RNA, proteins, bacteria, viruses, and disease specific markers for a variety of pathogenic disease states including Health Associated Infections (MRSA, C. difficile, Vancomycin resistant enterococcus (VRE), Norovirus), Critical Infectious Diseases (MTB/RIF, Flu, RSV, EV), Sexual Health (CT/NG, GBS), oncology (e.g., breast or bladder cancer) and Genetics (FIL/FV). In some embodiments, the system 10 can identify the type of cartridge via integrated near field communication ability (e.g. RFID, laser scanning), and thus apply the appropriate assay routine to the cartridge. In some embodiments, cartridge identification uses Bluetooth technology, RFID tags, barcoding, QR labels, and the like.

Once an assay cartridge is physically inserted within and initialized by the system 10, the system will perform the functions of specimen processing, which can in some embodiments include sample preparation, nucleic acid amplification, and an analyte detection process. Results of the detection process can be uploaded wirelessly or directly by wire to a local computer or cloud-based network. Advantageously, the local computer can be a wireless communication device, such as a tablet or cellular phone, having a software application specifically designed to control the system and communicate with a network.

The system 10 can be powered by an external power source, and can feature an uninterruptable power supply (e.g. batteries) in case of power disruption or field use. The uninterruptable power supply (UPS) allows for field use of the system, and in some embodiments can provide power to the system for at least one day, preferably up to two days. In some embodiments, the UPS allows for up to four hours of continuous operation. As shown in this external view, the system 10 can include an outer shell 12 and a door 14 for accepting an assay cartridge (not shown). Different styles of the outer shell 12 can be configured as needed by a particular user. Typically, outer shell 12 is formed of a substantially rigid material so as to protect and support the components within, for example, a hardened polymer or metal construction. Although not shown here, in some embodiments the outer shell 12 can be heavily ruggedized (armored) for field use, or as shown here made decorative for physician office use.

FIG. 1B shows an exploded view of the system 10 (without the outer shell) and with major subsystems depicted outwardly. An overview of the subsystems is provided below. Additional details of each subsystem are described in the following sections.

Various sub-systems are disclosed that make use of brushless DC (BLDC) motors. Generally, each motor can have a stator assembly that is mounted to a printed circuit board (PCB) substrate, and can include a backdrivable transmission mechanism, such as a lead screw. In some embodiments, such BLDC motors make use of analog sensors (e.g., Hall-sensors) for determining angular positioning and force-based current monitoring as a triggering tool. Such BLDC motors can include a rotor with multiple magnets disposed thereon and mounted to a stator on a substrate with at least as many sensors as phases of the motor. The three sensors are positioned such that the displacement of the rotor can be controlled based on the measurements from the sensors, thereby providing improved resolution and granularity without requiring use of any position-based sensors or encoder hardware. Thus, the BLDC motors described herein do not require use of encoder hardware and their associated drive trains do not require use of position sensors. For example, the system can include a syringe drive mechanism 16 that includes a brushless BLDC motor having an output shaft that is mated to a backdrivable lead-screw. The lead-screw drives a plunger rod that can interface with a plunger tip of a removable assay cartridge. Such a syringe drive mechanism 16 can share a PCB 30 with a door drive mechanism 18. The door drive mechanism also includes a BLDC motor having an output shaft that is mated to a backdrivable lead-screw. The motors of the syringe drive mechanism 16 and door drive mechanism 18 are shown directly mounted to opposite sides of a PCB board, however, this is not critical and both motors can be mounted to the same side. In some embodiments, each motor can be mounted to its own PCB. It is advantageous to utilize such BLDC motors as the improved resolution and granularity allows for improved accuracy and efficiency, and further allows for further miniaturization of mechanisms driven by such motors. It is appreciated, however, that use of such BLDC motors is not required and that any of the mechanisms described herein could also be driven by conventional type motors if desired, but additional sensors and/or circuitry may be required for some embodiments.

As mentioned above, the BLDC motor is unique in that it includes a plurality of Hall-effect sensors, but does not include any traditional encoder hardware extrinsic to the BLDC. In some embodiments, the syringe drive mechanism and door drive mechanism, and associated subsystems, do not include position sensors. In some embodiments, the angular position of the rotor and output shaft of the BLDC can be solely derived from the sinusoidal wave output of the analog sensors and the circuitry on the PCB. Thus, traditional position sensors (e.g. encoders, optical sensors, etc.) are not required for use in conjunction with the BLDC motors as used in the instant invention. In order for the BLDC motor to provide smooth torque production, motor control techniques such as sine-wave commutation can be implemented. Further, pulse-width modulation implementation can be used to achieve high speed operation with high electronic drive efficiency.

In addition, because the lead screws of the mechanisms are backdrivable, force-based end-of-travel detection can be used to determine start and stop points for driving the mechanisms. Force-based end-of-travel detection can be derived by monitoring the current of the BLDC motors, e.g., the current of a bridge circuit, which will deviate (increase or decrease) from a norm when a force-based event occurs. Hence, this deviation can be used as a trigger event to start, stop, reverse, slow down, and/or speed up a BLDC motor. For example, in the case of the syringe drive mechanism 16, drive current and voltage sensing can be correlated to pressure, and thus be used to deliver a consistent or intentionally varying pressure to the plunger rod by real-time adjustment of the BLDC motor speed. This alleviates the need for an in-line pressure sensor to monitor cartridge pressure.

Valve drive mechanism 20 can make similar use of the same type of BLDC motor. In some embodiments, the valve drive mechanism 20 can include a worm drive gear train, which ultimately outputs to a turntable-like valve drive for rotating the valve of a removable assay cartridge. In some embodiments, the worm drive mechanism is not backdrivable as in the aforementioned syringe drive and door drive mechanisms. However, the same type of Hall-effect position determination and force base triggering (current monitoring) can be used for the valve drive mechanism. For example, if turning the valve drive unexpectedly requires substantially less or more current, then such an event can be indicative of a jam or failure of an assay cartridge. Here, force base triggering can be used to sense a cartridge integrity malfunction.

Sonication horn mechanism 22 is partially integrated with the valve drive mechanism 20. The sonication horn mechanism 22 can apply a programmable sonication power for a programmable duration to the cartridge, for example, in order to lyse a target sample within the cartridge. In some embodiments, the sonication horn mechanism 22 can employ a resonant piezo-electric actuator to apply vibration at a frequency of about 30 kHz or greater, about 40 kHz or greater, such as about 50 kHz (e.g. 50.5 kHz). The sonication horn mechanism 22 includes a control circuit that uses the phase of measured current in relation to the voltage excitation to determine the resonant frequency. The frequency can be adjusted by the control circuit to maintain a preset phase relationship thereby tracking the resonance frequency as it changes during sonication. In some embodiments, the amplitude of the voltage excitation can be continually adjusted to maintain the commanded power level. Based on these functions, the control circuit can maximize power output of the horn in real-time.

The system 10 also includes a door drive and cartridge loading system 24 that is powered by the door drive mechanism 18. The lead screw of the door drive mechanism 18 outputs power to the door drive and cartridge loading system 24 to both open and close the door 14 as well as engage and intake an assay cartridge 32.

A rear chassis portion 26 and a front chassis portion 28 provide structural support for the system 10, as well as mounting provisions for the other subsystems. The chassis portions are generally elongated to provide a smaller overall footprint for the system 10 and enable portability of the system 10. In some embodiments, the system can have a foot print of: 9.1″×3.0″×4.2″, and an approximate weight of 2.2 lbs. The elongated circuit board or PCB 30 generally matches the foot print of the chassis portions. The PCB 30 includes most or all of the processors, sub-processors, memory, and control circuits required to control the system 10. However, the aforementioned BLDC motors can be integrated with their own respective printed circuit boards that have control circuits that connect separately to the PCB 30. The PCB 30 also includes communication circuit aspects (e.g. near field communication circuits, USB, wireless) as well as a power supply circuit.

The system 10 is compatible with various types of assay cartridges 32, which are generally configured for receiving and holding a sample of material, such as a bodily fluid (e.g., blood, urine, saliva) or solid (e.g., soil, spores, chemical residue) that is liquid soluble. The assay cartridge 32 can be a walled structure having one or more fluid channels and connection ports. The assay cartridge 32 may be relatively small, such that it can easily be hand-held, portable, and/or disposable. Examples of such cartridges (useable with the system 10) are disclosed in U.S. Pat. No. 6,660,228, Int'l Pub. No. WO 2014052671 A1, U.S. Pat. No. 6,374,684, which are each incorporated by reference herein for all purposes.

The assay cartridge 32 can include a reaction vessel 33 extending outward from the cartridge body, which interfaces with a thermal cycling and detection module 34. The module 34 includes one or more apparatuses configured to deliver energy to, and also remove energy from, an aspect of the assay cartridge 32. Such an apparatus can include a dual thermoelectric cooler. The module 34 also includes one or more detection aspects, as discussed in further detail below.

### II. Brushless DC (BLDC) Motor Architecture

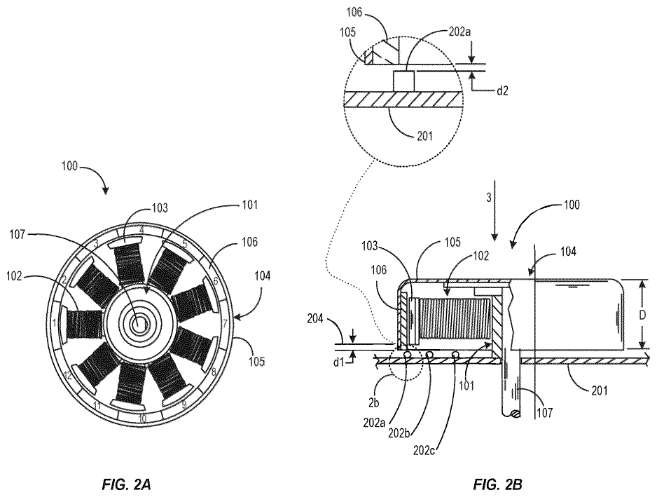

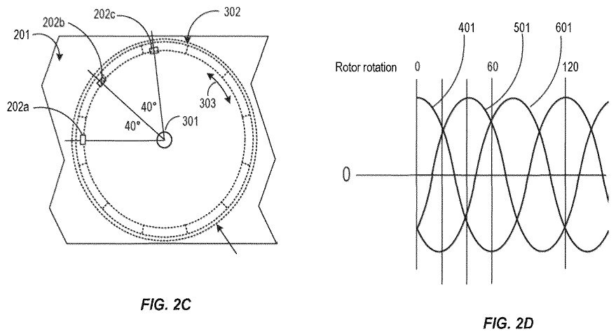

FIG. 2A is a plan view diagram illustrating elements of a brushless DC (BLDC) motor 100, for use with some embodiments of the invention. Further details of the BLDC motor can be U.S. Pat. No. 10,348,225 entitled “Encoderless Motor with Improved Granularity and Methods of Use” issued Jul. 9, 2019, and U.S. Provisional patent application Ser. No.______ entitled “Encoderless Motor with Improved Quantization and Methods of Use and Calibration” filed concurrently herewith; each of which is hereby incorporated by reference for all purposes.

In one aspect, the BLDC motor includes a rotor and a stator configured to produce a smoothly-varying Hall-effect voltage without any need for filtering or noise reduction. In some embodiments, this feature is provided by use of permanent magnets within the rotor that extend a distance beyond the magnetic core of the stator. In some embodiments, the BLDC motor includes as many Hall-effect sensors as phases of the motor, which are positioned such that the motor can be controlled based on the measured voltage patterns received from the sensors. In some embodiments, this includes spacing the sensors radially about the stator such that the measured voltage waveforms intersect. For example, a three-phase BLDC can include three Hall-effect sensors spaced 40 degrees radially from each other, thereby allowing the system to control a position of the sensor within an increment of 40 degrees.

In some embodiments, the motor comprises an internal stator assembly 101 having nine pole teeth extending radially from center, each pole tooth ending in a pole shoe 103, and each pole tooth having a winding providing an electromagnetic coil 102. The motor further comprises an external rotor 104 having an external cylindrical skirt 105 and twelve permanent magnets 106 arranged with alternating polarity around the inner periphery of the skirt 105. The permanent magnets are shaped to provide a cylindrical inner surface for the rotor with close proximity to outer curved surfaces of the pole shoes. The BLDC motor in this example is a three-phase, twelve pole motor. Controls provided, but not shown in FIG. 2A, switch current in the coils 102 providing electromagnetic interaction with permanent magnets 106 to drive the rotor, as is well-known in the art.

It should be noted that the number of pole teeth and poles, and indeed the disclosure of an internal stator and an external rotor are exemplary, and not limiting in the invention, which is operable with motors of a variety of different designs.

FIG. 2B is a side-elevation view, partly in section, of the motor of FIG. 2A, cut away to show one pole tooth and coil of the nine, ending in pole shoe 103 in close proximity to one of the twelve permanent magnets 106 arranged around the inner periphery of cylindrical skirt 105 of external rotor 104. The pole teeth and pole shoes of stator assembly 101 are a part of the core and define a distal extremity of the core at the height of line 204. Stator assembly 101 is supported in this implementation on a substrate 201, which in some embodiments is a printed circuit board (PCB), comprising controls and traces for managing switching of electrical current to coils 102, providing electromagnetic fields interacting with the fields of permanent magnets 106 to drive the rotor. The PCB as substrate can also comprise control circuitry for encoding and commutation. Rotor 104 engages physically with stator assembly 101 by drive shaft 107, which engages a bearing assembly in the stator to guide the rotor with precision in rotation. Drive shaft 107 in this implementation passes through an opening for the purpose in PCB 107 and can be engaged to drive mechanical devices.

Three linear Hall-effect sensors 202a, 202b, and 202c are illustrated in FIG. 2B, supported by substrate 201, and positioned strategically according to some embodiments of the invention to produce a variable voltage pattern that can be used in a process to encode angular position of the rotor and provide commutation for motor 100. In FIG. 2B the overall height of skirt 105 of rotor 104 is represented by dimension D. Dimension d1 represents extension of the distal extremity of the rotor magnets below the distal extremity of the core at line 204. In conventional motors there is no reason or motivation to extend this edge below the extremity of the core, particularly since this can increase the height of the motor and require increased clearance between the rotor and substrate. In fact, the skilled artisan would limit dimension D so there is no such extension, as the added dimension would only add unnecessary cost and bulk to a conventional motor. Furthermore, in conventional motors at the distal extremity of the rotor, at the height of or above the distal extremity of the core, switching of current in coils 102 creates a considerable field effect, and a signal detected by a Hall-effect sensor placed to sense permanent magnets at that position would not produce a smoothly varying Hall-effect voltage. Rather, the effect in a conventional motor is substantially noise corrupted. The conventional approach to this dilemma is to introduce noise-filtering, or more commonly to utilize an encoder.

Extending the rotor magnets below the distal extremity of the iron core avoids the corrupting effect of the switching fields from the coils of the stator on the signal detected by the Hall-effect sensors. The particular extension d1 will depend on several factors specific to the particular motor arrangement, and in some embodiments will be 1 mm or more (e.g. 2 mm, 3 mm, 4 mm, 5 mm, 6 mm, or greater), while in some embodiments the extension will be less than 1 mm. In some embodiments, the distance is a function of the size of the permanent magnets and/or the strength of the magnetic field. In some embodiments, as detailed herein, 1 mm of extension is sufficient to produce a sinusoidal signal of varying voltage without noise or saturation. Placement of the Hall-effect sensors at a separation d2 to produce a Hall-effect voltage produces a smoothly variable voltage, devoid of noise. In some embodiments, the Hall-effect sensors produce a smoothly variable DC voltage in the range from about 2 volts to about 5 volts devoid of noise or saturation. The dimension d2 may vary depending on choice of sensor, design of a rotor, strength of permanent magnets in the rotor, and other factors that are well known to persons of skill in the art. A workable separation is readily discovered for any particular circumstance, to avoid saturation of the sensor and to produce a smoothly variable DC voltage substantially devoid of noise.

FIG. 2C is a plan diagram of a portion of substrate 201 taken in the direction of arrow 3 of FIG. 2B, showing placement of Hall-effect sensors 202a, 202b, and 202c relative to the distal edge of rotor 104, which may be seen in FIG. 2B to extend below the distal edge of the core by dimension d1. In FIG. 2C the rotation track of rotor 104 including the twelve permanent magnets 106 is shown in dotted outline 302. The rotor rotates in either direction 303 depending on details of commutation.

As illustrated in this non-limiting exemplary embodiment, each of Hall-effect sensors 202a, 202b, and 202c is positioned radially beneath the distal edge of the rotor magnets, just toward the inside of the central track of the rotating magnets. Hall-effect sensor 202b is located forty degrees arc from Hall-effect sensor 202a along the rotating track of the magnets of the rotor. Similarly, Hall-effect sensor 202c is located a further forty degrees around the rotor track from Hall-effect sensor 202b.

FIG. 2D illustrates three voltage patterns 401, 501 and 601 produced by passage of permanent magnets 106 of rotor 104 over Hall-effect sensors 202a, 202b, and 202c in a three-phase BLDC motor. A sinusoidal variable voltage pattern 401 produced by passage of permanent magnets 106 of rotor 104 over Hall-effect sensor 202a. The 0 degree starting point is arbitrarily set to be at a maximum voltage point. Three complete sine waveforms are produced in one full 360 degree revolution of the rotor. Voltage pattern 501 produced by passage of permanent magnets 106 of rotor 104 over Hall-effect sensor 202b. Further, a substantially noise free sinusoidal variable voltage pattern 501 produced by passage of permanent magnets 106 of rotor 104 over Hall-effect sensor 202b. As Hall-effect sensor 202b is positioned at an arc length of 40 degrees from the position of Hall-effect sensor 202a, sinusoidal pattern 501 is phase-shifted by 120 degrees from that of sinusoidal pattern 401. Yet further, a substantially noise free sinusoidal variable voltage pattern 601 produced by passage of permanent magnets 106 of rotor 104 over Hall-effect sensor 202c. As Hall-effect sensor 202c is positioned at an arc length of 40 degrees from the position of Hall-effect sensor 202b, sinusoidal pattern 601 is phase-shifted by 120 degrees from that of sinusoidal pattern 501. The patterns repeat for each 360 degree rotation of the rotor.

The three voltage patterns 401, 501 and 601 each have substantially the same max and min peaks, as the Hall-effect sensors are identical, and are sensing the same magnetic fringe fields at the same distances. Moreover, patterns 401, 501 and 601 intersect at multiple points, points 402, 502, and 602 being examples, as shown in FIG. 2D. Because the physical rotation of the rotor, in this example, from one pattern intersection to another is twenty degrees of motor rotation, each voltage change by the calculated amount then represents 20/20, that is, 1.00 degrees of rotation of the rotor. This is a relatively gross example to merely illustrate the method. In some embodiments, the motor displacement can be determined and controlled from these signals. In one aspect, the control unit can determine motor displacement from the signals without performing error correction or filtering of individual signals and without a hardware encoder or dedicated positional sensor. In some embodiments, the control unit combines the sensor signals by performing a transformation matrix of the three signals which avoids any second order effects that may affect individual signals during motor operation. This approach is described in further detail in U.S. patent application Ser. No.______. In this implementation the mechanical rotational translation of the rotor for each count is about 0.0098 degree. Resolution of the system can be increased (or decreased) by using an ADC with a higher (or lower) bit resolution. For example, using an 8-bit ADC would resolve each count to about 0.078 degrees, a 16-bit ADC would resolve each count to 0.00031 degrees, and using a 20-bit ADC would resolve each count to about 0.00002 degrees. Alternatively, increasing or decreasing the number of poles will correspondingly increase or decrease the resolution of the system.

In some embodiments, the invention provides for a high degree of accuracy and precision for mechanisms driven by motor 100. In the non-limiting example described above using an 11-bit ADC, the motor position can be controlled to 0.0098 degree mechanical. Coupled with gear reduction extremely fine control of translation and rotation of mechanisms can be attained. In some embodiments, motor 100 is coupled to a translation drive for a syringe-pump unit to take in and expel fluid in diagnostic processes.

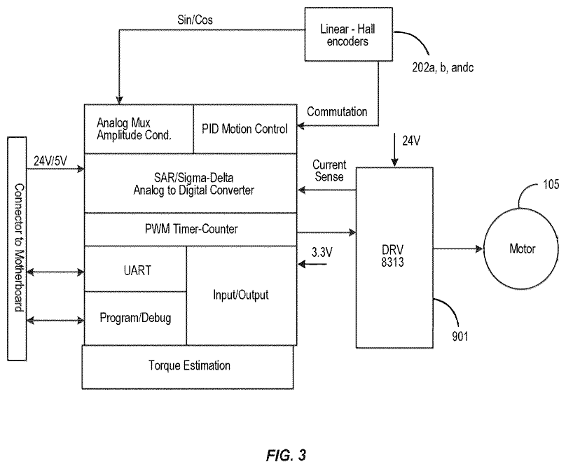

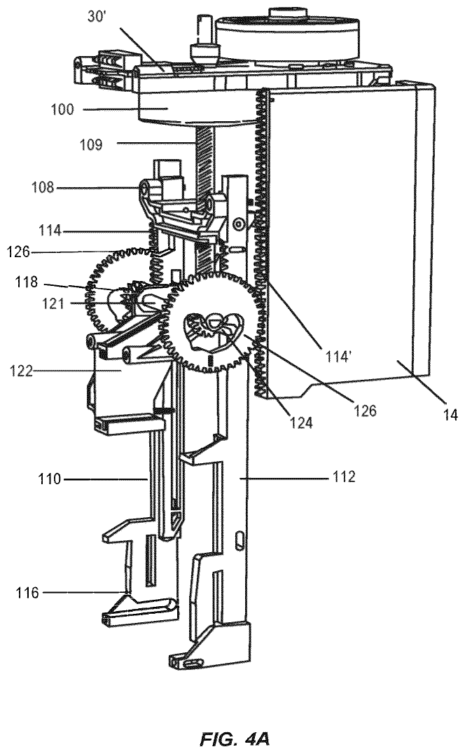

FIG. 3 is a diagram depicting circuitry in some embodiments of the invention for controlling motor 100 using the output of the Hall-effect sensors and the unique method of processing the phase-separated curves produced by the sensors, as described above. The decoded position determined from the Hall-effect sensors 202a, 202b, and 202c is provided for commutation purpose, and the waveforms produced by interaction of the rotor magnets with the Hall-effect sensors is provided to multiplexer circuitry as shown in FIG. 3. The decoded position is also fed to proportional-integral-derivative (PID) motion control circuitry to control the position in accordance to a real-time commanded position. As described above in the non-limiting exemplary embodiments, an ADC is used to produce the division of the straight portions of the phase-separated waveforms and motor 100, which can be driven by, for example, a DRV8313 Texas Instruments motor driver circuit. The skilled person will understand the circuitry is not necessarily unique and will understand further that there are other arrangements of circuitry that might be used while still falling within the scope of the instant invention. In some embodiments the circuitry and coded instructions for sensing the Hall-effect sensors and providing motor encoding can be implemented in a programmable system on a chip (PSoC) on the PCB. The circuitry can also include a torque estimating circuit, which can be provided to estimate torque values generated by the motor based on current and voltage measurements taken at the PSoC, thus avoiding the need for additional force sensors throughout the greater system.

### III. Door Opening and Cartridge Loading Sub-system

In another aspect, the invention provides a door opening/closing and cartridge loading sub-system that is driven by a backdrivable mechanism so as to facilitate ease in manual loading and unloading an assay cartridge from the diagnostic assay system. In some embodiments, the door opening/closing mechanism and cartridge loading system are integrated so as to provide coordinated movement such that manual loading of the cartridge into an open bay of the system initiates closing of the bay door, typically upon detection of backdriving of the mechanism as the user manually pushes the cartridge into the system. It is appreciated that such mechanisms can be driven by a BLDC motor, as described herein, and utilize motor torque estimation, or utilize various conventional motors and approaches as would be known to one of skill in the art. Examples of such configurations are detailed below.

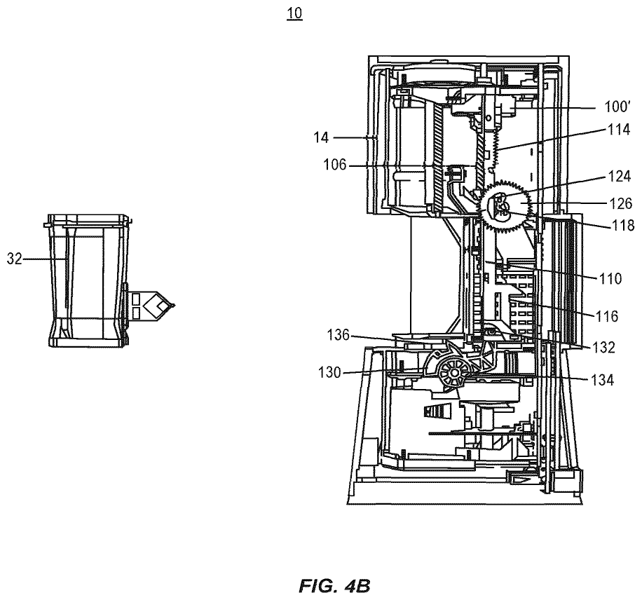

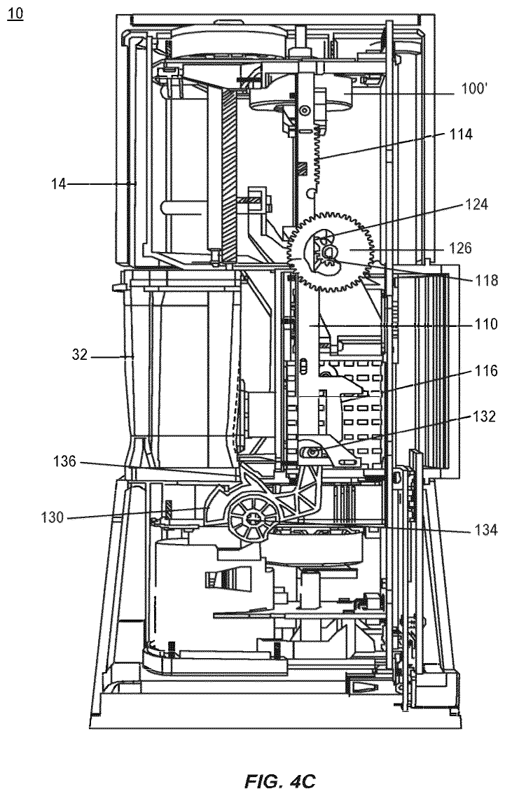

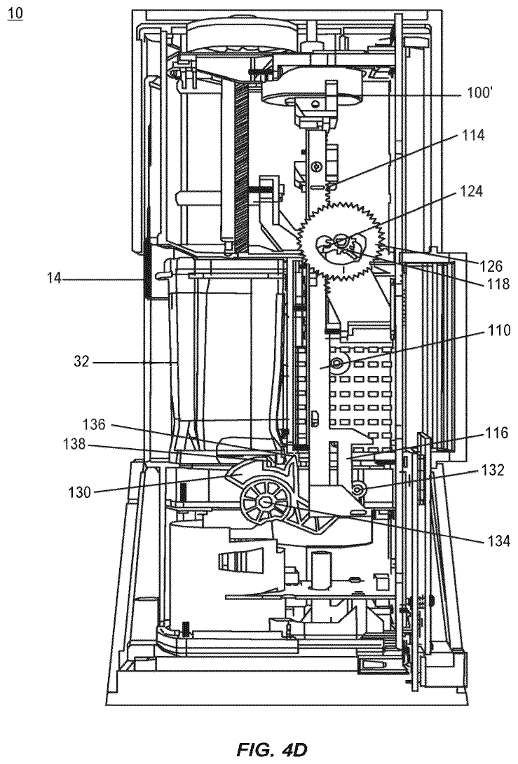

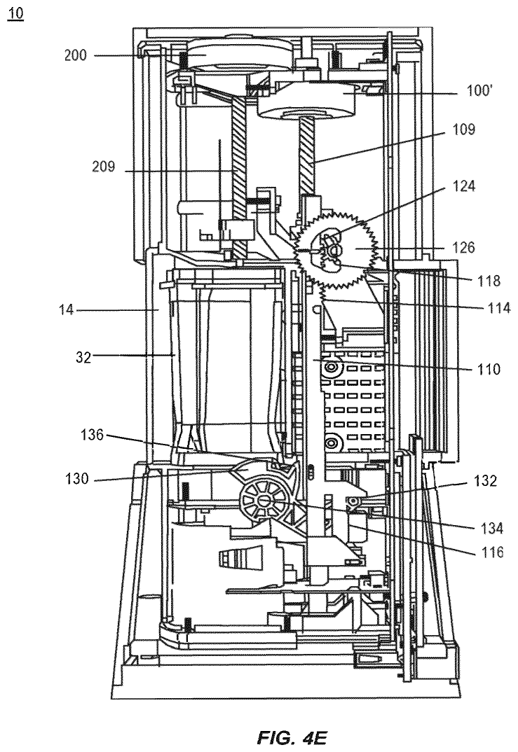

FIG. 4A shows a perspective view of a door opening and cartridge loading sub-system 100. The system includes a brushless DC (BLDC) motor 100, as described above, mounted to a PCB 30′. The BLDC motor 100 includes an output shaft (not shown) to which a lead screw 109 is attached. The lead screw 109 is back drivable aspect of a transmission that operates to open and close the door 14 as well as power a cartridge loading mechanism.

The lead screw 109 threads engage with a nut of a bridge 108, hence, when the lead screw 109 turns, the bridge 108 moves upward or downward (as the device is oriented in FIG. 4A) depending on the direction the lead screw 109 turns. A first rack portion 110 and a second rack portion 112 are affixed to the bridge 108. Both rack portions are elongated to include a rack 114 and a cam pathway 116, that forms an “L” like path.

A pair of pinion gears 118 are meshed with the racks 114. Up and down movement of the racks 114 is caused by movement of the bridge 108 and the lead screw 109, which causes the pinions 118 to rotate accordingly. The pinion gears 118 are connected to each other by a shared shaft 121 that is supported by a sub-frame 122, which is affixed to a greater portion of the system 10, such as rear chassis portion 26. Each pinion gear 118 includes a finger 124 for stopping rotation of the pinion gear 118 at certain interfaces.

Each pinion gear 118 is integrated with a larger door gear 126. Accordingly, the pinion gears 118 and door gears 126 spin at the same RPM. The door gears 126 interfaces with door racks 128 of the door 14. Hence, when the door gears 126 turn, the door racks 128 and door 14 move up or down according to the direction the door gears 126 are spinning.

FIGS. 4B-4E graphically depict a method of loading an assay cartridge. At FIG. 4B, a command is sent to a BLDC motor 100′ to open the door 14 to place the system into position to accept insertion of the cartridge 32. When the command is received, the system 100 operates the BLDC motor 100′ to turn the lead screw 109. This action causes the bridge 108 and affixed rack portions 110/112 to move upwardly, and hence initiate turning of the pinion gears 118 and door gears 126. This movement will cause the door 14 to travel upward as the door gears 126 spin against the door racks 128.

After the door 14 is completely open, the pinion gears 118 disengage from the racks 114 of the first and second rack portions 110/112, which continue to move upwards. Upward movement of the first and second rack portions 110/112 also causes cartridge loading arms 130 to be actuated by the pins 132 that are constrained to move along the cam pathways 116 of the first and second rack portions 110/112. The cartridge loading arms 130 are forced by this movement to spin about pivots 134, which places first arm portions 136 into an upward position.

The first and second rack portions 110/112 will move upwardly, until a force-based event occurs that back drives the lead screw 109. Such an event can be, for example, the bridge 108 encountering a stop or the first and second rack portions 110/112 pulling against the cartridge loading arms 130. The backdriving event can be detected at a bridge circuit of the BLDC motor as a change in current. Based on the backdriving event, the BLDC motor is commanded to stop turning and rest in the position shown. Advantageously, this step is performed without the aid of any position sensors.

At FIG. 4C, the assay cartridge 32 is inserted into the system 10 until a portion of the assay cartridge 32 is brought into contact with the first arm portions 136. Slight movement against the first arm portions 136 results in another backdriving event at the lead screw 109 that is detectable at the bridge circuit of the BLDC motor as a change in current. This event serves as a command for the BLDC motor to reverse direction from the previous door-opening step in order to capture the cartridge and close the door.

As shown at FIG. 4D, upward movement of the first and second rack portions 110/112 causes the pins 132 to be guided about the length of the cam pathways, which in turn causes the cartridge loading arms 130 to rotate in a clockwise direction. This causes second arm portions 138 of the cartridge loading arms 130 to push the cartridge inward into a home position. In addition, the first and second rack portions 110/112 are raised until the fingers 124 of the pinion gears 118 are turned by notches 140 of the first and second rack portions 110/112, which initiates movement of the pinion gears 118 against the rack 114, as well as the door gears 126 against the door rack 114, which has teeth 114′ that interact with the door gears 126. In this manner, the door 14 is made to travel downward towards a closed position.

As shown at FIG. 4E, the door 14 is made to travel downward by continued movement of the lead screw 109 to completely close the door. The BLDC motor is powered to do so until a force-based event occurs that back drives against the lead screw 109. Such an event can be, for example, the bridge 108 encountering a stop or the first and second rack portions 110/112 pushing against the cartridge loading arms 130. The backdriving event can be detected at the bridge circuit of the BLDC motor as a change in current. Based on detection of the backdriving event, the BLDC motor is commanded to stop turning and rest in the position shown. Advantageously, this step is performed without the aid of any position sensors.

### IV. Syringe Drive Sub-System

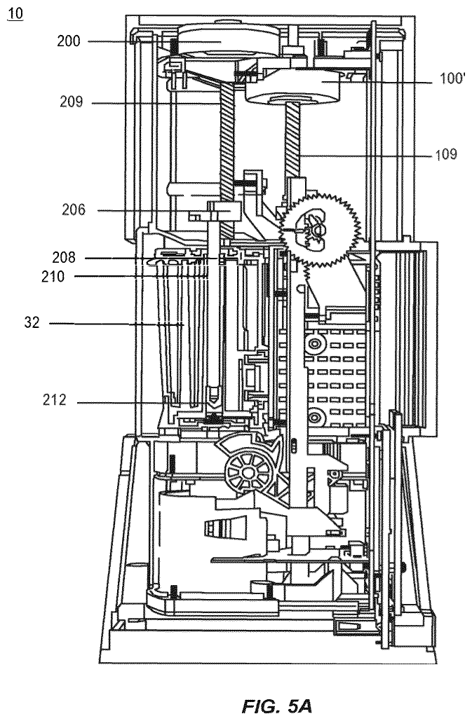

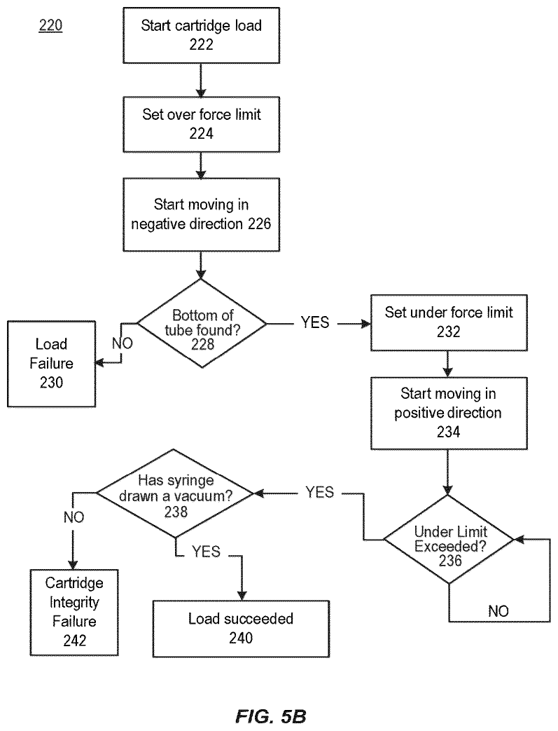

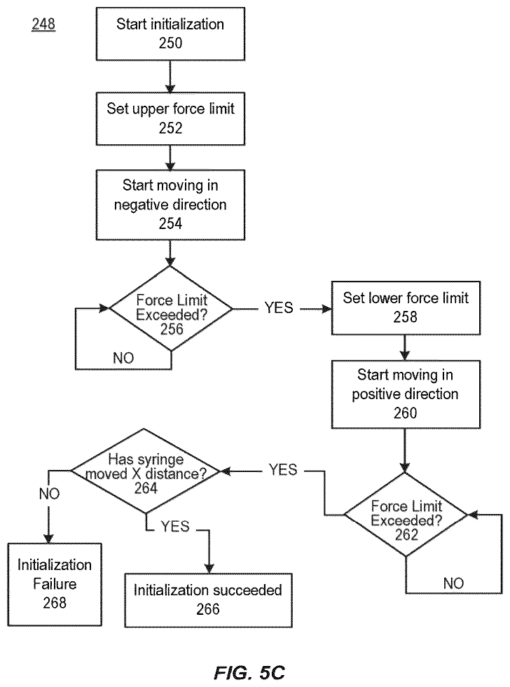

As described above, embodiments of the invention can include aspects of the syringe drive mechanism 16. As shown at FIG. 5A, the syringe drive mechanism 16 includes a BLDC motor 200 as described above. The BLDC motor 200 includes an output shaft that is connected to a backdrivable lead screw 209.

A laterally extending arm 206 includes a nut that is threaded to the lead screw 209. The laterally extending arm 206 also is affixed to a plunger rod 208. The laterally extending arm 206 and plunger rod 208 can be driven downward and upward by commanding the BLDC motor 200 to turn the lead screw 209 in an appropriate direction.

After the assay cartridge 32 is secured and the door 14 is closed, the syringe drive mechanism 16 can be utilized to interface with the assay cartridge 32. The assay cartridge includes a syringe passage 210 holding a plunger rod 208 having a plunger tip 212. Downward movement of the plunger rod 208 into the syringe passage 210, which causes the tip of the plunger rod 208 to engage the plunger tip 212. In this manner, the combined plunger tip 212 and plunger rod 208, together with the syringe passage, functions as a syringe to pressurize/depressurize the assay cartridge 32. Programmed pumping of the assay cartridge 32 causes fluid to flow into and out from various chambers of the assay cartridge 32 to affect an assay.

After engagement with the plunger tip 212, the plunger rod 208 can be actuated by the BLDC motor 200 to any desired position within the syringe passage 210, including enactment of various syringe pumping algorithms. BLDC motor 200 drive voltage and current can be continually monitored to determine the plunger rod pressure alleviating the need for an in-line pressure sensor to monitor cartridge pressure.

Accordingly, because the lead screw 209 can be backdriven, a pressure decrease within the assay cartridge 32 can cause a stationary plunger rod 208 to be pulled downward. The pressure decrease can be detected by monitoring the measured current of the BLDC motor 200, detecting a relative change, and then changing the output of the BLDC motor 200 accordingly. Similarly, a pressure increase within the assay cartridge 32 can cause a stationary plunger rod 210 to be pushed upward. The pressure increase can be detected by monitoring the measured current of the BLDC motor 200, detecting a relative change, and then changing the output of the BLDC motor 200 accordingly. Advantageously, this can be performed without the aid of any pressure sensors.

In another example, the current associated with a moving plunger rod 208 can be monitored for changes that indicate increases or decreases in pressure rate. Hence, after detecting a relative change, the output of the BLDC motor 200 can be changed to increase or decrease the pressure rate being applied by the moving plunger rod 208. Advantageously, this can be performed without the aid of any pressure sensors.

An example of a method 220, using the aforementioned principles of BLDC current monitoring, for determining proper loading of an assay cartridge and testing integrity of that cartridge is depicted at FIG. 5B. It is assumed that the assay cartridge 32 has been already physically loaded as shown at FIG. 5A.

At operation 222, a command is sent to begin the loading procedure. As a result, an over force limit is set at operation 224. The over force limit is the maximum force the BLDC motor 200 may exert onto the plunger rod 208 for the purposes of this operation, which is associated with the plunger rod 208 compressing the plunger tip 212 against the bottom of the syringe passage 210. At operation 226, the BLDC motor 200 is operated to move the plunger rod 208 into the syringe passage 210, which causes the tip of the plunger rod 208 to engage the plunger tip 212. At operation 228 torque of the BLDC motor 200 is continually monitored, using the torque estimation circuit of FIG. 2E and the methodology of FIGS. 3A-3C, to determine if the plunger rod 208 has travelled to the bottom of the syringe passage 210. If the over force limit is not exceeded then it is determined that the bottom of the syringe passage has not been found and so that the loading procedure has failed at operation 230. Occasionally, the plunger tip 212 may be missing due to a manufacturing error or physically deficient. In either case, the plunger rod 208 will meet the end of its possible travel with the syringe passage 210 without properly bottoming against a plunger tip 212, and hence, the over force limit will not be exceeded.

If the over force limit is exceeded then it is determined that the plunger rod 208 has pushed the plunger tip 212 to the bottom of the syringe passage 210, and the method 220 moves to operation 232, where an under-force limit is set. The under-force limit is the maximum force the BLDC motor 200 may exert onto the plunger rod 210 for the purposes of this operation, which is related to decompressing the plunger tip 212. At operation 234 the BLDC motor 200 is operated to move the plunger rod 210 upward within the syringe passage 210. At operation 236 torque of the BLDC motor 200 is continually monitored to determine if the under limit has been exceeded. As a result of operation 228, the plunger tip 212 will be highly compressed. The under limit is the amount of force required to decompress the plunger tip and thereby zero out the position of the plunger tip 212 for later operation. Once the under limit is exceeded, the BLDC motor 200 will cease operation and the method will move to operation 238, where it is determined if the syringe has drawn a vacuum. At this operation, valving of the assay cartridge 32 is operated to seal off the syringe passage 210 to atmosphere, which was not the case in the preceding steps. After this is complete, the BLDC motor 200 is operated to pull the plunger rod 208 upwards against the vacuum within the syringe passage 210. If the plunger rod 208 does not move freely and force is detected, then at operation 240 it is determined that vacuum has been established and thus integrity of the assay cartridge 32 is not comprised. If the plunger rod 208 moves freely without detection of force, then at operation 242 it is determined that no vacuum has been established and thus integrity of the assay cartridge 32 is compromised.

Another example of a method 248, using the aforementioned principles of BLDC current monitoring, for determining initializing the syringe of the assay cartridge (i.e., plunger rod 208, syringe passage 210, and plunger tip 212) is depicted at FIG. 5C. It is assumed that the assay cartridge 32 has been already physically loaded as shown at FIG. 5A, and the cartridge has been loaded properly as shown at FIG. 5B.

At operation 250, a command is sent to begin the loading procedure. As a result, an upper force limit is set at operation 252. The over force limit is the maximum force the BLDC motor 200 may exert onto the plunger rod 208 for the purposes of this operation, which is associated with placing the plunger tip 212 at a proper upward position (relative to the orientation of the device as shown in FIG. 5A) at the top of the syringe passage 210.

At operation 254, the BLDC motor 200 is operated to move the plunger rod 208 upwardly within the syringe passage 210, which causes the plunger tip 212 to top out at a position within the syringe passage 210. At operation 256 torque of the BLDC motor 200 is continually monitored, using the torque estimation circuit of FIG. 2E and the methodology of FIGS. 3A-3C.

Once the over-force limit is exceeded then it is determined that the plunger tip 212 is topped out, and the method 248 moves to operation 258, where a lower force limit is set. The lower force limit is the maximum force the BLDC motor 200 may exert onto the plunger rod 210 for the purposes of this operation, which is related to placing the plunger tip 212 against the bottom of the syringe passage 210, but without excessive compression of the plunger tip 212. At operation 260 the BLDC motor 200 is operated to move the plunger rod 210 downwardly within the syringe passage 210. At operation 262, torque of the BLDC motor 200 is continually monitored to determine if the lower force limit set at operation 258 has been exceeded. Once the lower limit is exceeded, the BLDC motor 200 will cease operation, and it is assumed the plunger tip 212 has been placed at the bottom of the syringe passage 210. After this, the method 248 will move to operation 238, where it is determined if the syringe has moved a predetermined amount of distance (e.g. 60 mm). This is performed by using the Hall-effect sensors of the BLDC motor 200 to count revolutions of lead screw 209 and relating that count to an amount of linear travel of the syringe rod 208. In some cases the upper and lower force limits will be triggered by obstructions or excessive friction within the syringe passage 210. Hence, the travel check step is performed to make sure the plunger rod 208 (i.e. syringe) has moved freely without obstruction. If the syringe rod 208 has moved at least the predetermined amount of travel, then it is determined that initialization is successful at operation 266. However, if the syringe rod 208 has not moved at least the predetermined amount of travel, then it is determined that initialization is not successful at operation 268.

### V. Valve Drive Sub-System

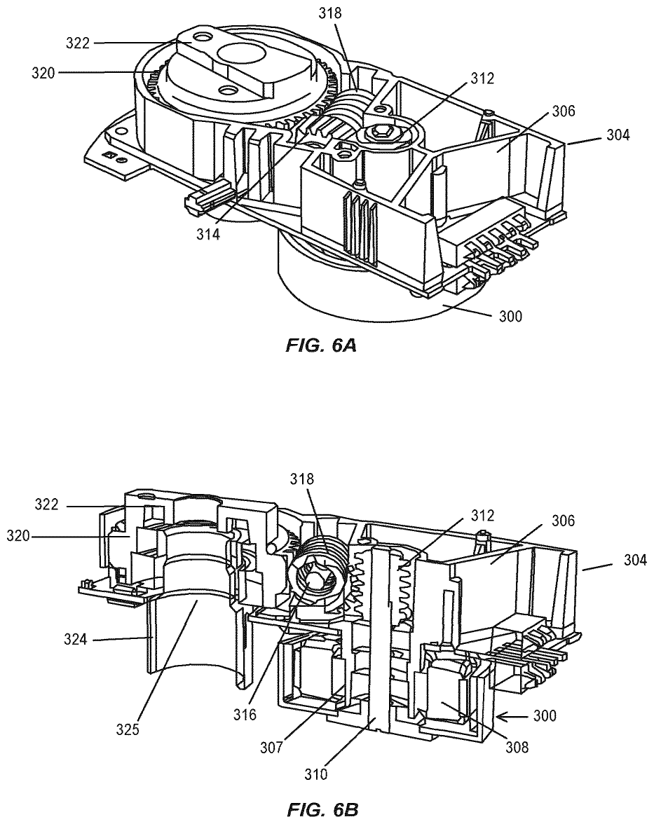

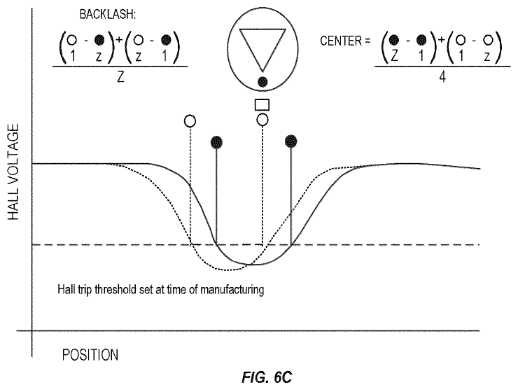

As described above, embodiments of the invention can include aspects of the valve drive mechanism 20. As shown at FIGS. 6A and 6B, the valve drive mechanism 20 includes a BLDC motor 300 as described above.

The BLDC motor 300 is mounted to a chassis 304 having a plurality of reinforcing ribs 306 that contribute to the rigidity of the chassis 304. The chassis 304 includes an elongated first portion 307 that serves as a mount for a stator 308 of the BLDC motor 300. An elongated shaft 310 extends from the BLDC motor 300 and holds a first worm 312. The first worm 312 cooperates with and turns a first worm gear 314, which turns on a shaft 316 shared with a second worm 318.

The second worm 318 cooperates with and turns a second worm gear 320. The second worm gear 320 is integrated with a turntable like valve drive 322, which is configured to cooperate with a turning valve mechanism of the assay cartridge 32. The valve drive 322 is mounted to an elongated second portion 324 of the chassis 304. The elongated second portion 324 includes a passage 325 for cooperation with the sonication horn mechanism 22.

In use, the BLDC motor 300 is powered to turn and thereby turns valve drive 322 via the worm drives described above. The valve drive 322 is substantially geared down, which allows for great precision when positioning the valve drive 322. The syringe drive mechanism 16 does not include any position sensors, because angular position of the stator 308 can be solely derived from the sinusoidal wave output of the Hall-effect sensors that measure the displacement of the rotor magnet poles, and through that position of the valve drive by knowledge of the final drive gear ratio.

The worm drives are not backdrivable as in the aforementioned syringe drive and door drive mechanisms. However, the same type of Hall-effect position derivation and force-based triggering can be used for the valve drive mechanism. Here, force base triggering can be indicative of a cartridge integrity malfunction. For example, if turning the valve drive unexpectedly requires substantially less or more power, then such an event can be indicative of a jam or failure of an assay cartridge. While each of the syringe drive, door drive mechanisms and valve drive mechanisms are described as utilizing the improved BLDC motor described herein, it is appreciated that any or all of the drives and mechanisms could also utilize a conventional type BLDC motor, a servo motor or other suitable motor, as would be understood by one of skill in the art, however some features may require additional sensors or circuitry.

In addition, the BLDC motor is configured to home and center position of the valve drive output by performing a centering protocol based on the sinusoidal signal generated by the Hall-effect sensors. This can compensate for gear backlash and gear wear over time. This is illustrated by the Hall voltage signal to valve drive position graph shown at FIG. 6C. As shown, a given position of the valve drive 322 can vary according to gear backlash and wear.

### VI. Sonication Horn Subassembly

In some embodiments, an ultrasonic horn subassembly is provided for use in an diagnostic assay system as described herein. In some embodiments, the ultrasonic horn assembly includes an ultrasonic horn, a horn housing, a spring, a chassis and control circuitry configured for operation of the horn. The horn housing is adapted for supporting and securing the ultrasonic horn and includes a section for retaining a spring coil to facilitate movement between a disengaged and engaged horn position and a wedge for interfacing with a cam mechanism of the system to actuate movement of the horn between the disengaged (lowered) and engaged (raised) positions. Although a coil spring is described herein, it is appreciated that various other types of springs or biasing mechanisms can be used. In the disengaged position, the tip of the ultrasonic horn is flush or below a base surface upon which the assay cartridge sits to facilitate loading and removal of the assay cartridge from the system. In the engaged position, the tip of the ultrasonic horn extends above the base surface so as to engage a domed portion of a sonication chamber of the assay cartridge to facilitate sonication of biological material in a fluid sample contained within the sonication chamber during sample analysis preparation and/or processing. In some embodiments, the movement of the horn is effected by an actuator mechanism common to one or more other movable components of the system, such as a door of the system. The horn assembly also includes circuitry, such as a printed circuit board, with interfaces adapted for electrical connection to corresponding circuitry within the system to facilitate operation of the ultrasonic horn by the system.

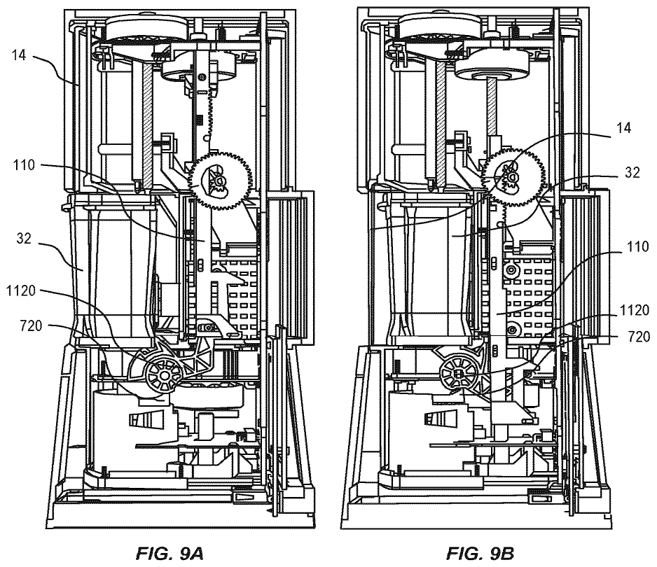

In some embodiments, the diagnostic assay system is placed upright during performance of an assay (as shown in FIGS. 9A-B) such that the horn moves between the disengaged position (lowered below the cartridge) and the engaged position (raised toward the cartridge) so as to engage and contact the sonication chamber of the cartridge. It is appreciated that in some embodiments, the design could be different such that in the disengaged positions and engaged positions the horn could be in various other orientations and/or locations relative the cartridge depending on the design of the cartridge and the diagnostic assay system.

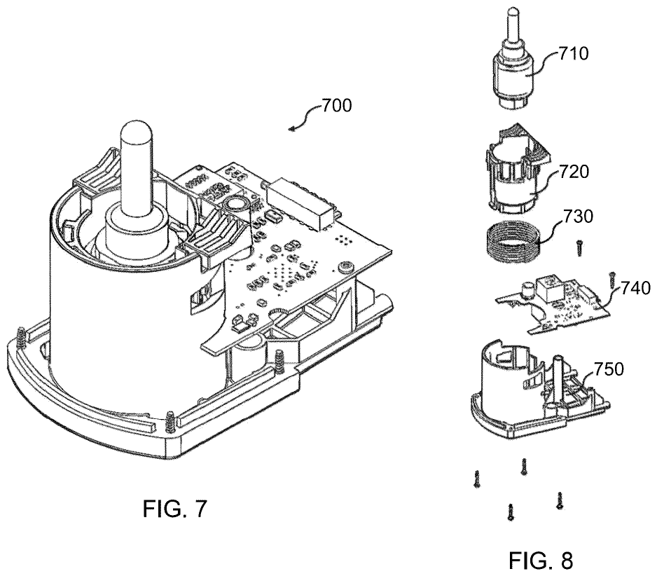

FIG. 7 illustrates an ultrasonic horn subassembly 700 configured for use in a diagnostic assay system in accordance with some embodiments of the invention. FIG. 8 depicts an exploded view of the horn assembly of FIG. 7. In this embodiment, the horn subassembly includes an ultrasonic horn 710, horn housing 720, spring coil 730, control circuitry 740, and chassis 750. The horn subassembly can be tested as a stand-alone sub-assembly before insertion into the system and may also be removed or replaced as needed.

The ultrasonic horn 710 snaps into the horn housing 720 (shown cut-away to show the horn residing within). The housing can be designed such that snapping the horn into the housing locates or clocks the horn within a pre-determined orientation and position relative the housing. For example, the ultrasonic horn can be of a design that includes features that are not perfectly axi-symmetric about a longitudinal axis of the horn such that corresponding features or surfaces on an interior portion of the housing engage to secure the horn into position within the housing and inhibit rotation of the horn therein. The non-axisymmetric feature may include, but is not limited to, a flat portion on one or both sides of the horn or a protrusion or tab extending outwardly from the horn or a contact through which the horn is electrically connected. In some embodiments, the horn 720 is incorporated into the subassembly and controlled with the control circuitry to provide an output suitable for lysing biological materials as needed for a particular assay.

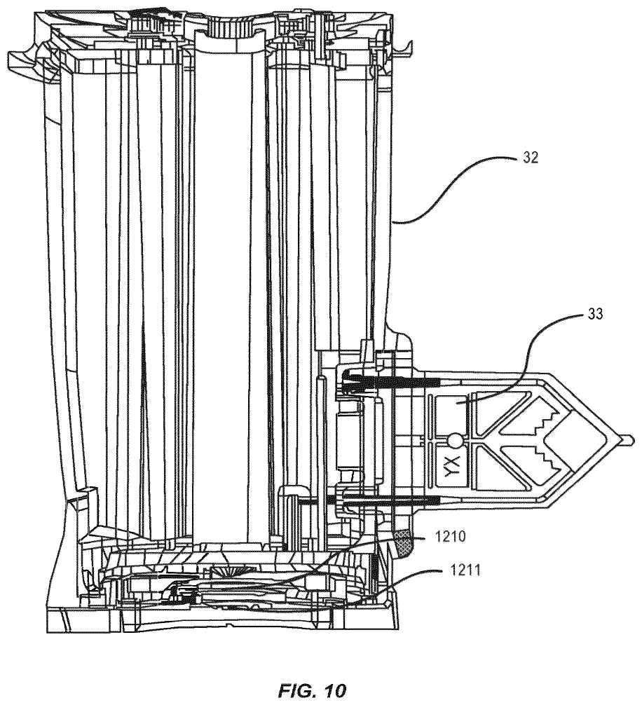

In some aspects, the ultrasonic horn is mounted on a movable mechanism by which the ultrasonic horn is positioned relative to a sonication chamber of an assay cartridge disposed within a diagnostic assay system. In some embodiments, the assay cartridge includes a sonication chamber positioned on the bottom of the cartridge (as oriented in FIG. 10) with a downward facing dome (outer surface of the dome being convex shaped with respect to the assay cartridge), as shown in the example of FIG. 10, that corresponds to a rounded tip 711A of the domed output portion 711 of the ultrasonic horn. Although the tip is rounded in this embodiment, it is appreciated that the tip of the dome portion may be shaped in a variety of shapes, including but not limited to flat, pointed, concave, convex, rounded, or domed, as desired. The dome shaped portion of the sonication chamber and the rounded horn tip focus the ultrasonic energy transmitted from the horn so as to efficiently reach the desired ultrasonic levels required to lyse cellular material (e.g. ruggedized cell, spores, etc.) and release nucleic acids contained therein into the fluid sample with minimal ultrasonic horn power and size requirements. Although an interfacing cam and wedge are described herein, it is appreciated that various other mechanisms may be used with or without a biasing member to facilitate movement of the horn between the disengaged and engaged positions. For example, in some embodiments, such mechanisms can include a lead screw, cable, and the like.

In some embodiments, the movable mechanism by which the ultrasonic horn is positioned to press against the sonication chamber is integrated within an inter-connector network of actuators that effect movement of various other components of the diagnostic assay system, such as opening and closing of a door of the system, loading and ejection of the assay cartridge from the system, movement of a valve assembly and a syringe assembly within the system. It is appreciated that the movable mechanism may be integrated with actuators of one or more other components or the movable mechanism may be entirely independent of other mechanisms and actuators.

FIGS. 9A-9B illustrates cross-sectional views of a diagnostic assay system during and after loading of an assay cartridge into the system demonstrating a mechanism that positions the ultrasonic horn in coordination with closing of a door of the system and loading of the assay cartridge. FIG. 9A depicts a partially inserted assay cartridge 32 in which a distal facing portion of a base of the assay cartridge begins to engage an ejection tooth of an ejection/loading cam 1120. In this position of the cam 1120, the outer surface of the cam engages an upper surface 721 of the wedge portion 721 of the horn housing.

As the assay cartridge 32 is more fully inserted, the assay cartridge presses against the ejection tooth and the ejection/loading cam 1120 rotates clock-wise so that a loading tooth of the cam engages an underside surface of the assay cartridge pulling the cartridge inward to a fully loaded position. As the ejection/loading cam 120 rotates the outer surface 1121 of the cam slides along the wedge tip 721a of the wedge portion 721 of the horn housing slide, which presses the horn housing away from the cartridge to the disengage position, which partly compresses the spring coil 730. As the assay cartridge is fully inserted, the wedge tip 721a is received within an inwardly curved portion 1121a of the rounded portion of the cam 1120 that allows the horn housing 720 to move upward a short distance allows the coil to at least partly decompress such that the rounded tip 711a of the ultrasonic horn protrudes above the surface along which the assay cartridge was loaded and pressingly engages the dome-shaped portion of the sonication chamber. As can be seen in FIGS. 9A and 9B, rotation of the cam 1120 is actuated by a closing movement of the first rack portion 110 of the door rack mechanism, which in this embodiment is downward movement (in the direction of the arrow). Through a network of interrelated gears, this closing movement of the door also simultaneously actuates closing of the door 14 of the system 1000 from an open position in FIG. 9A to facilitate insertion and loading of the assay cartridge 32 to a closed position, as shown in FIG. 9B, after loading of the cartridge. Movement of the door rack mechanism can be effected by one or more motors, such as any of those described herein.

FIG. 10 illustrates a cross-sectional view of an assay cartridge for use in a diagnostic assay system in accordance with some embodiments of the invention. The dome-shaped portion 1211 of the sonication chamber 1210, described above, is positioned on the bottom surface of the assay cartridge. The sonication chamber 1210 is in fluid communication with a network of channels in the assay cartridge, through which fluid is transported by movement of a valve and syringe to effectuate pressure changes during the assay procedure. After the sample is prepared and/or processed, the prepared fluid sample is transported into a chamber of the reaction vessel 33, while an excitation means and an optical detection means are used to optically sense the presence or absence of a target analyte (e.g. a nucleic acid) of interest (e.g., a bacteria, a virus, a pathogen, a toxin, or other target analyte). It is appreciated that such a reaction vessel could include various differing chambers, conduits, micro-well arrays for use in detecting the target analyte. An exemplary use of such a reaction vessel for analyzing a fluid sample is described in commonly assigned U.S. Pat. No. 6,818,185, entitled “Cartridge for Conducting a Chemical Reaction,” filed May 30, 2000, the entire contents of which are incorporate herein by reference for all purposes.

### VII. Motor Torque/Force Estimation

In some embodiments, aspects of the BLDC motor and control circuits can be used to sense motor torque or force to facilitate fine-tuned operation of a mechatronic system, such as a syringe drive, valve drive, cartridge loader/unloader or door opening/closing system of the diagnostic assay module described above. In conventional modules, torque estimation can be accomplished in different ways, for example by estimating torque based on the principle that the electrical power put forth into the BLDC motor is equal to the mechanical power extracted from the motor in addition to the electrical power dissipated by the motor (i.e. copper loss). This principal is quantified by the following equations:

$${P}_{i n} = {P}_{out} + {P}_{C L}$$

Where dissipated power P_{CL} is calculated from:

$${P}_{CL} = \frac{3}{2} {i}_{q}^{2} {r}_{m}$$

$${P}_{CL} = \frac{3}{2} \frac{{r}_{m}}{{K}_{t}^{2}} {τ}_{m}^{2} or {P}_{CL} = {α}_{CL} {τ}_{m}^{2} , with {α}_{CL} = \frac{3}{2} \frac{{r}_{m}}{{K}_{t}^{2}}$$

Referring to the power balancing equation above, it logically follows that:

$$0 = {P}_{out} + {P}_{CL} - {P}_{i n}$$

Substitution of the power variables results in the following balanced equation:

$$0 = ( {α}_{CL} * {τ}_{m}^{2} ) + ( {ω}_{m} * {τ}_{m} ) - ( {v}_{B} * {i}_{B} )$$

Hence, solving for the motor torque τₘ, the following equation results:

$${τ}_{m} = \frac{- {ω}_{m} \pm \sqrt{{ω}_{m}^{2} - 4 {α}_{C L} {v}_{B} {i}_{B}}}{2 {α}_{C L}}$$

It follows that here are two possible calculated solutions for the motor torque, which are the most positive and most negative torque solutions generated by the preceding equation, using bridge current i_{B}, as shown below:

$${\hat{τ}}_{m 1} = \frac{- {ω}_{m} + \sqrt{{ω}_{m}^{2} - 4 {α}_{{CL}^{{v}_{B} {i}_{B}}}}}{2 {α}_{CL}} and {\hat{τ}}_{m 2} = \frac{- {ω}_{m} - \sqrt{{ω}_{m}^{2} - 4 {α}_{{CL}^{{v}_{B} {i}_{B}}}}}{2 {α}_{C L}}$$

Given that torque is calculable from the motor constant and other variables, the motor torque can also be calculated using the motor constant Kₜ.

$${\hat{τ}}_{m} = {K}_{t} {i}_{q} ≅ {K}_{t} \frac{{v}_{q} - {v}_{E M F}}{\frac{3}{2} {r}_{m}} , where {V}_{E M F} = {K}_{t} {ω}_{m}$$

Thus, in the conventional approach, the calculated solution \hat{τ}ₘ₁ or \hat{τ}ₘ₂ that is closest to the calculation for \hat{τ}ₘ (using Kₜ) is assumed to be the correct solution. The following table defines the variables above.

| Variable | Notation | Details |
|---|---|---|
| Bridge Voltage | v_{b} | The DC bus voltage supplied to the |
| | | motor drive power electronics |
| Bridge Current | i_{b} | The current supplied to the motor drive |
| | | power electronics by the bus voltage |
| Low-pass filter | f_{B} | The bandwidth in Hz of the low-pass |
| bandwidth | | filters employed in the force computation |
| Discrete Time Sample | Tₛ | The interval between the samples in the |
| Period | | discrete time control system. |
| Motor Torque | Tₘ | The motor torque applied to the rotor by |
| | | the stator windings |
| Motor Velocity | ωₘ | The angular velocity of the motor |
| Motor Torque solutions | \hat{τ}ₘ_{₁}, \hat{τ}ₘ_{₂} | The most positive and most negative |
| | | torque solutions generated by the Motor |
| | | Torque Solution Algorithm |
| q, d components | ( )_{q}, ( )_{d} | The component of voltage or current that |
| | | aligns with the torque-producing, q, and |
| | | non-torque-producing, d, vectors that |
| | | denote the q, d coordinate system. |
| Motor Electrical | ωₑ | The motor electrical frequency—a value |
| Frequency | | equal to the product of the number of |
| | | $pole ‐ pairs , \frac{{N}_{p}}{2} and the motor angular$ |
| | | velocity, ωₘ |
| Motor Constant | kₜ | The motor constant that determines the |
| | | scaling relationship between the motor |
| | | torque and motor current (τₘ = kₜi_{q}) and |
| | | between the motor voltage and motor |
| | | angular velocity (v_{q} = kₜωₘ). |
| Motor voltage | v_{g}, v_{d} | The vector that defines the motor voltage |
| | | within the (q, d) coordinate system |
| Motor current | i_{q}, i_{d} | The vector that defines the motor current |
| | | within the (q, d) coordinate system |
| Motor Winding Voltage | V_{A}, V_{B}, V_{G} | The voltages applied by the three-phase |
| | | inverter to the motor windings |
| EMF Voltage | v_{emf} | The back-emf (electro-motive force) is |
| | | the open-circuit voltage generated when |
| | | rotating the motor rotor, v_{emf} = kₜωₘ |
| Estimated or computed | (\hat{ }) | This refers to the computed value, |
| value | | including filtered signal representations. |
| | | Lack of the “\hat{ }” designation refers to the |
| | | actual value prior to sensing. |
| Motor resistance | rₘ | This is the winding resistance as |
| | | measured from output to “center tap” |
| | | (CT), which is a contact made at a |
| | | halfway point along the winding. |

The principles above can be relied on for estimating torque values based on the readily available current and voltages measurements. While this conventional approach is sufficient in many cases, the estimate torque or forces in some instances may not be accurate, particularly when bridge current was low. In addition, there are additional variables, such as friction and second order effects (e.g. harmonics) that can degrade accuracy of the estimated and resulting control of the mechatronic system. Variation can be as much as +/−75% when all error sources are taken into account. The following describes alternative embodiments for estimating motor torque or force, which can be used for pressure or force sensing to improve accuracy of control of an associated mechatronic system. These approaches can be achieved by utilizing control units having a processor and memory with instructions having computing instructions and control algorithms recorded thereon for controlling operation of the mechatronic system in accordance with the concepts described herein. These control units are achievable using a low-cost Programmable System-on-Chip integrated circuit, such as the PSoC® line of circuits available from Cyprus Semiconductor Corp

In one aspect, the invention pertains to an improved approach to determining motor torque or force, which can be utilized in various mechatronic systems, including the syringe drive, valve drive, cartridge loader/unloader and door opening/closing systems of the diagnostic assay module described herein. It is appreciated that the methods described herein can be implemented in firmware of a control unit that operates any of the above noted mechatronic systems.

### A. Pressure Sensing

In regard pressure sensing, the methods can incorporate this aspect into different procedures, including any of: pressure estimation, pressure calibration, pressure verification, cartridge integrity testing and self-testing. The approaches described herein are advantageous in regard to estimating pressure as it accounts for transmission characteristics. The syringe transmission characteristics includes motor, motor drive and transmission friction. These approaches can also be utilized to provide syringe transmission calibration to allow for syringe operation with greater accuracy. In some embodiments, these improved methods can be implemented in a conventional diagnostic assay module without changing the PCBA hardware, position control firmware and the CLOAD command (e.g. “stop on pressure” as used in tube bottom finding).

Accurate pressure sensing is needed for various reasons. First, in regard to the CLOAD, it is important that the syringe system accurately locate the syringe bottom-stop position so that aspiration/dispensing volumes are accurate. Secondly, accurate pressure sensing can be used to determine cartridge integrity by identifying and rejecting “leaky” cartridges prior to running an assay. Third, accurate pressure sensing can be used to abort processing when a pressure problem is identified (e.g. patient sample too viscous, valve-port misaligned).

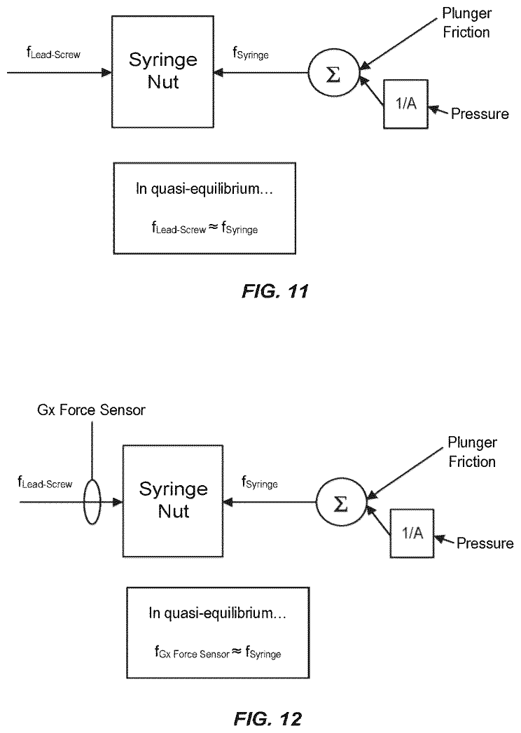

FIG. 11 depicts a simple force balance approach to pressure sensing, which relies on the quasi-equilibrium on a portion of the actuator, in this embodiment, the lead-screw force and the syringe force at the syringe nut. FIG. 12 depicts a similar approach but further includes a force sensor along the lead-screw so that this force can be measured directly, thereby improving accuracy.

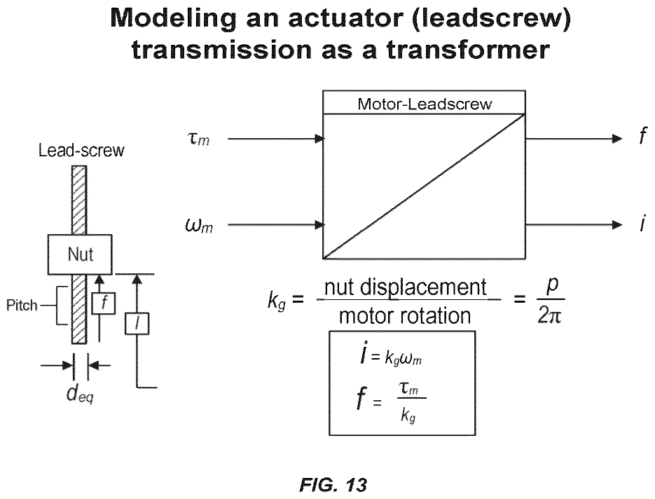

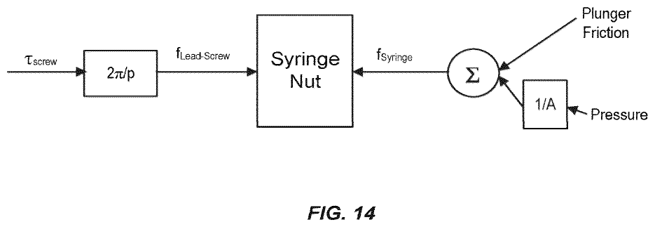

FIG. 13 is a schematic that illustrates modeling of an actuator transmission (e.g. leadscrew) as a transformer. FIG. 14 depicts the corresponding control diagram in which the force is sensed by estimating the “effort” to turn the leadscrew τ_{screw}.

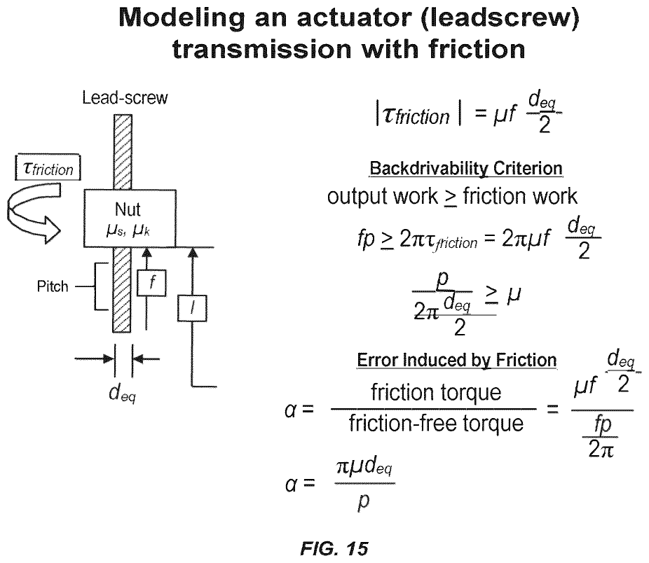

FIG. 15 is a schematic that illustrates modeling of an actuator transmission (e.g. leadscrew) that further accounts for friction. FIG. 16 depicts the corresponding control diagram in which the force is measured by estimating the “effort” to maintain equilibrium. This approach utilizes drag torque and load-dependent friction torque as inputs. One drawback with this approach is that the measurement of the effort is confounded by load-dependent friction and drag.

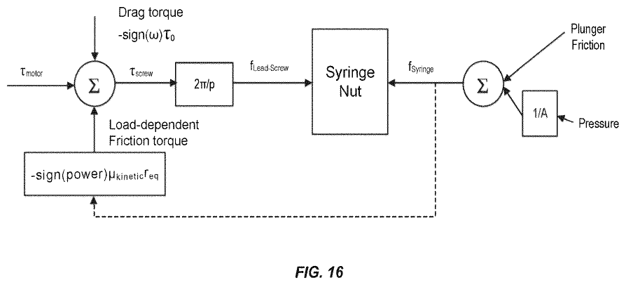

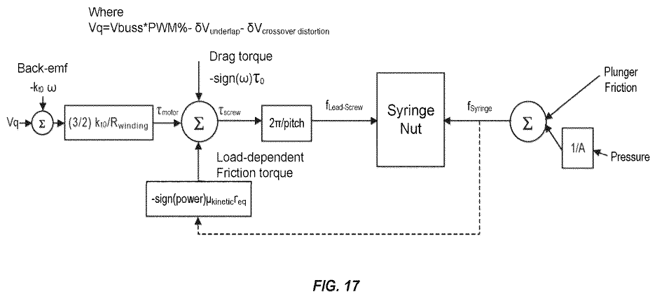

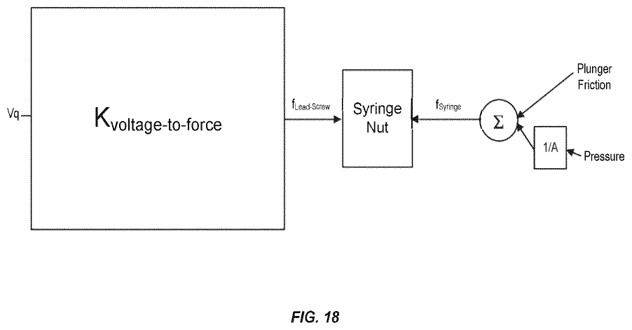

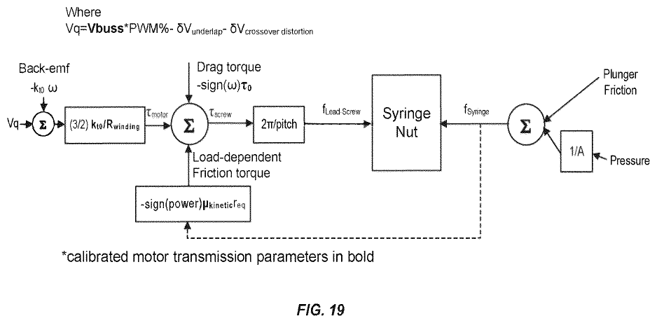

FIG. 17 is a control diagram in which the estimate of motor torque depends on the applied voltage as determined by the product of the bus voltage motor PWM %; the power supply bus voltage, the motor speed, winding resistance (T), motor constant and motor driver “distortion.” This approach additionally utilizes back-emf as an input. In contrast, FIG. 18 depicts a previous approach that modeled the transmission as an uncalibrated scale factor and ignored the friction, temperature-dependent and non-linear effects. As noted above, in the conventional approach, variation can be as much as +/−75% when all error sources are taken into account. FIG. 19 is a control diagram that illustrates an improved approach in accordance with some embodiments. This approach utilizes calibrated motor transmission parameters (bolded/blue) to estimate the force.

Advantageously, the approaches described above allow for estimating syringe pressure in real-time. In some embodiments, a filter can be used to account for second order effects, including electrical cycle harmonics or acceleration effects. These approaches also allow for pressures sensing that account for transmission characteristics, including those of the motor, the motor drive and the transmission. Characteristics of the motor include winding resistance, Rm(Tw), motor constant, K_{To}. Characteristics of the motor drive include bridge voltage, V_{buss}, PWM Underlap, delta V(nominal), and cross-over distortion, and δV(nominal). Transmission characteristics include lead-screw coefficient-of-friction, μₖ and running friction, τ₀.

i. Pressure Estimation

As shown in FIG. 19, pressure estimation can be improved by utilizing calibrated motor transmission parameters. In some embodiments, the following calculations can be used to sense motor torque. The first equation is a V_{q} calculation from the three PWM using an inverse Clark transform:

$$[ \begin{matrix}{V}_{q} Sin θ \\ {V}_{q} Cos θ\end{matrix} ] = {V}_{Bridge} ⋆ [ \begin{matrix}\frac{2}{3} & \frac{- 1}{3} & \frac{- 1}{3} \\ 0 & \frac{\sqrt{3}}{2} & \frac{- \sqrt{3}}{2}\end{matrix} ] ⋆ [ \begin{matrix}p w {m}_{1} \\ {pwm}_{2} \\ {pwm}_{3}\end{matrix} ]$$

$${V}_{q} = \sqrt{{V}_{q} Sin {θ}^{2} + {V}_{q} Cos {θ}^{2}}$$

$$Clark Transform = [ \begin{matrix}\frac{2}{3} & \frac{- 1}{3} & \frac{- 1}{3} \\ 0 & \frac{\sqrt{3}}{2} & \frac{- \sqrt{3}}{2}\end{matrix} ]$$

Resistance Measurement can be Determined from the Following Equation:

$${r}_{m} = \frac{3}{2} \frac{{V}_{q} ( {V}_{q} - k {T}_{0} {ω}_{m} )}{{I}_{Bridge} {V}_{bridge}}$$

### Where:

rₘ=MotorResistance
K_{T0}=TorqueConstant
ωₘ=Velocity
V_{q}=2φ Velocity
V_{Bridge}=BridgeVoltage
I_{Bridge}=BridgeCurrent
   Estimated Torque can be Determined from the Following Equation:

$${r}_{m} = \frac{3}{2} {K}_{T 0} \frac{{V}_{q} ( {V}_{q} - k {T}_{0} {ω}_{m} )}{{r}_{m}}$$

### Where:

rₘ=MotorResistance
K_{T0}=Torque Constant
ωₘ=Velocity
V_{q}=2φ Velocity
τₘ=Motor Torque
   The Lead Screw Force can be Derived from the Following Equation:

$${f}_{s} = \frac{{τ}_{q} - sign ( {ω}_{m} {τ}_{m} ) {τ}_{0} ( \frac{2 π}{p} )}{( 1 + sign ( {ω}_{m} {τ}_{m} ) ( \frac{2 π}{p} ) μ {r}_{screw} )}$$

### Where:

fₛ=Net force delivered by screw
τ₀=Unloaded Friction Torque
μₛ=Coefficient of Friction of the screw
r_{screw}=Effective radius of the screw
p=Lead Screw pitch
ωₘτₘ=power delivered

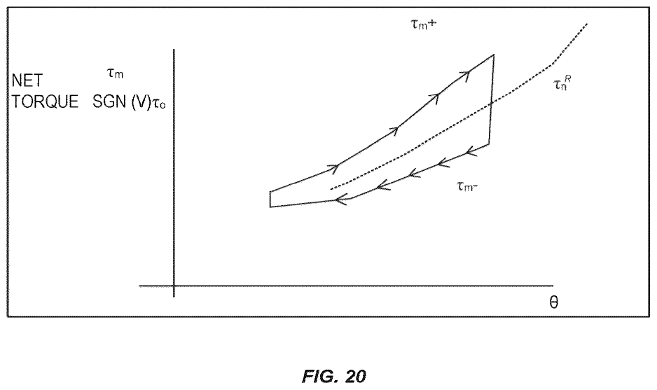

The following approach can be used to determine the coefficient of friction. FIG. 20 illustrates a plot of torque versus displacement. Again, the mechanical work method over delta theta=n 2πₑ can be used, as in the following equations:

$$\begin{matrix}∝ = [ {\int }_{{θ}_{0}}^{{θ}_{0} + Δ θ} ( {τ}_{μ}^{+} - S G N ( {V}^{+} ) {τ}_{θ} ) {d}_{θ} / {\int }_{{θ}_{0} + Δ θ}^{{θ}_{0}} ( {τ}_{\overline{μ}} - S G N ( {V}^{-} ) {τ}_{θ} ) {d}_{θ} ] \\ = \frac{1 + SGN ( {V}^{+} ) ( \frac{2 π}{P} ) {r}_{screw} {\hat{μ}}_{s}}{1 + SGN ( {V}^{-} ) ( \frac{2 π}{p} ) {r}_{screw} {\hat{μ}}_{s}}\end{matrix} \qquad ( 1 )$$

### Setting

$$β = ( \frac{2 π}{P} ) {r}_{screw}$$

The α term is the ratio of the mechanical work while extending to the mechanical work while retracting at the same extension. Thus, the first equation can be written as:

$$( 1 - β {μ}_{s} ) ∝ ( 1 + β {μ}_{s} )$$

From this equation, the estimate of the coefficient-of-friction can be determined from the following equation:

$${S}_{0} \qquad ( 2 )$$

$${\hat{μ}}_{s} = ( \frac{∝ + 1}{∝ - 1} ) β$$

$$where β = ( 2 π / p ) ⋆ req ≅ 1$$

The computation of coefficient of Friction (μₛ) can help screen any component variation (e.g. motor, alignment, fabrication/mount) and reject any assembly with higher than threshold value. The coefficient of friction can be determined from the following equation.

$${μ}_{s} = ( \frac{a + 1}{a - 1} ) β$$

### Where:

$${μ}_{s} = ⌈ \frac{{\int }_{{θ}_{0}}^{{θ}_{0} + Δ θ} ( {τ}_{m}^{+} − sgn ( {V}^{+} ) {τ}_{θ} ) {d}_{θ}}{{\int }_{{θ}_{0} + Δ θ}^{{θ}_{0}} ( {τ}_{m}^{−} − sgn ( {V}^{−} ) {τ}_{θ} ) {d}_{θ}} ⌉$$

$$\hat{{τ}_{0}} = \frac{1}{2} [ {\hat{T}}_{m} {\vert}_{{I}_{v + ve}} - {\hat{T}}_{m} \vert {I}_{v - ve} ]$$

### Where:

\hat{τ}_{m|I}_{ᵥ₊ᵥₑ}=Motor Torque in positive direction
\hat{τ}_{m|I}_{_{v−ve}}=Motor Torque in negative direction
   and

$$β = ( \frac{2 π}{P} ) {r}_{s c r e w}$$

where
Lead Screw Pitch=6.35 mm
r_{screw}=1000 mm

$$∴ ( \frac{2 * π}{P} ) {r}_{screw} ∼ 1$$

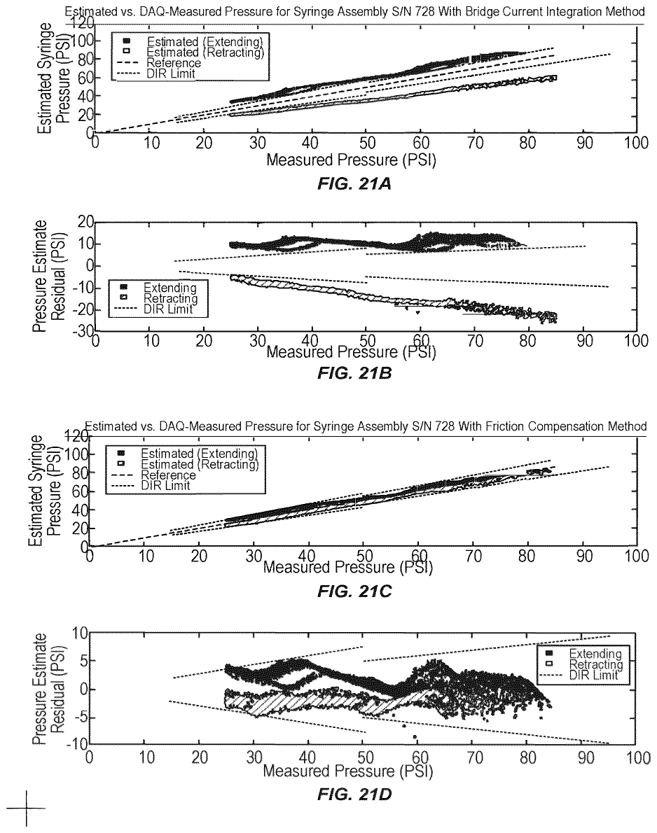

FIGS. 21A-D illustrate estimated syringe pressure (PSI) versus measured pressure (PSI), which illustrate the effects of friction. FIG. 21A depicts a conventional integration method. FIGS. 21A and 21B depict pressure estimation error, which demonstrate lack of accuracy of the estimate when the motor kt and friction compensation are not applied. The motor kt and friction compensation method described herein is shown in FIGS. 21C and D, which demonstrates significantly higher degree of accuracy in the estimate.

In regard to friction artifacts, the term us is the notation for coefficient of friction. Conventional pressure sensing techniques of the module do not compensate for μK. In regard to pressure calibration setup, the same setup can be used in accordance with the improved pressure sensing approaches described herein. In some embodiments, an automated calibration process is introduced by utilizing a specialized calibration instrument in place of the cartridge. In some embodiments, an additional calibration is performed for pressures below 25 PSI.

In another aspect, the methods can include estimating the motor winding resistance. Motor resistance (e.g., winding resistance) is a major component of force measurement. There is also a temperature dependence as the resistance of the winding is a linear function of temperature, as shown by the following equation (in the equations below, RTC is for a copper wire at room temperature, it is appreciated that different wire compositions will have different scaling):

$$= {r}_{0} ( 1 + {R}_{T C} ( - {T}_{0} ) )$$

### Where:

T=current temperature
T₀=Nominal Temperature at which the winding resistance is known

$${R}_{T C} = Rate of Change of Resistance = 0.39 \frac{\%}{° C .}$$

r₀=Resistance of the winding at nominal temperature

### Winding Temperature (Tw) can Also be Estimated, for Example by the Following Equation:

$$= + [ \frac{{r}_{m 0}}{} - 1 ] / {R}_{T c}$$

### Where:

[symbol] =Current temperature of the windings
[symbol] =Current Resistance of the winding

$${R}_{Tc} = Rate of Change of Resistance = 0.39 \frac{\%}{° C .}$$

rₘ₀=Resistance of the winding at nominal temperature

Various additional aspects of the pressure sensing can be implemented in the firmware as well. For example, the V_{q}, Torque and Force estimation can be performed in the firmware of the system. The K_{To} and μₛ used can be obtained from the pressure calibration results. In regard to resistance measurements, this can refer to the mean resistance of multiple windings (e.g. three windings in a three-phase motor), and the voltage can be applied to one winding and the other two windings teed to the same potential. All the motor parameters (μₛ, K_{To}, mR) can be stored in the syringe control unit memory. In some embodiments, the VT Data vectors used can include torque, resistance, μₛ and pressure.

iii. Pressure Calibration

For calibration of pressure, an external load sensor and a data acquisition system (e.g. like the National Instrument DAQ) can be used to acquire force data from load sensor. The acquired force data can be saved in a log file (e.g. CellCoreVT log). Force data from syringe control unit can also be logged in the syringe VT log file. The load sensor is loaded where the cartridge would otherwise be loaded and the system performed dispensing/aspirating cycles while the sensor collects pressure data.

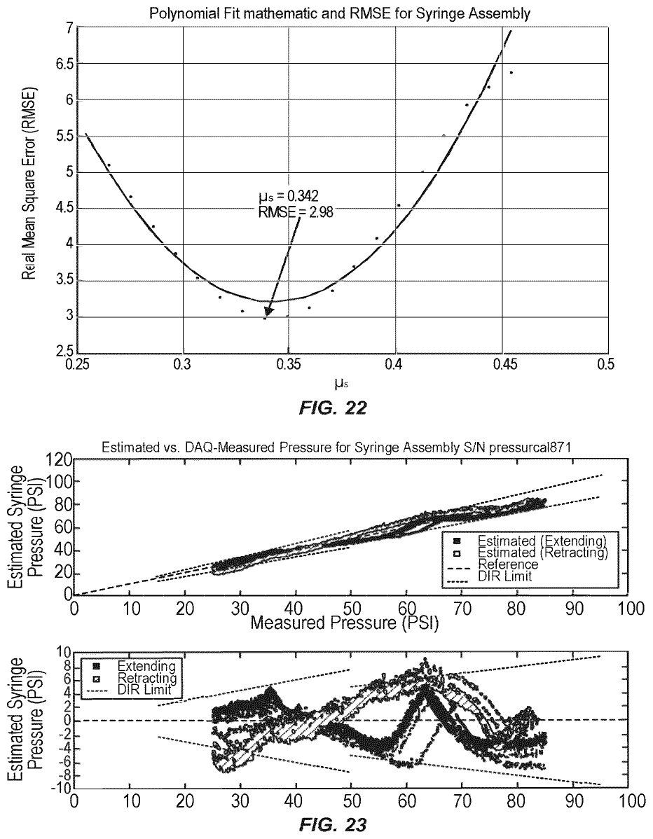

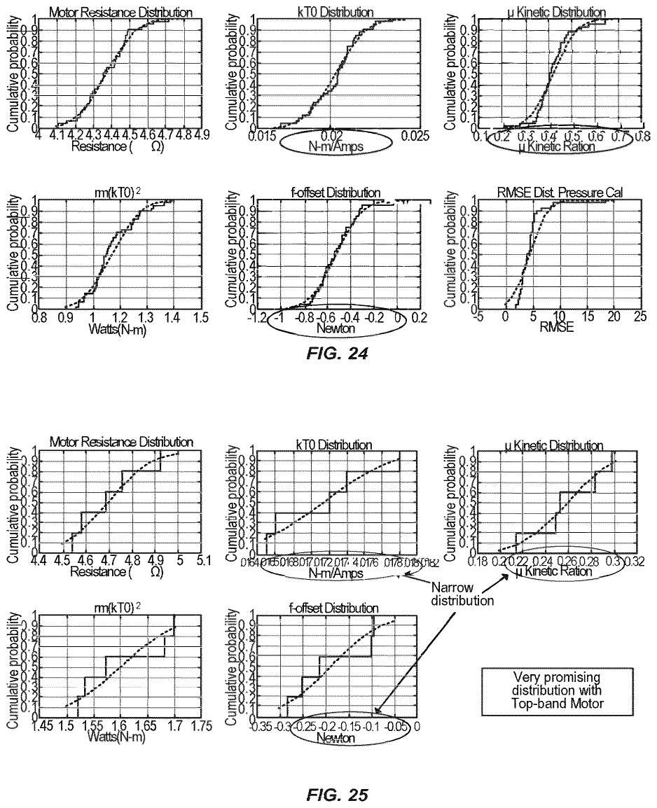

From the sensor data collected, parameters of the system can be estimated. For example, as shown in FIG. 22 a curve fit of the measured force data from the data acquisition system and syringe can be used to estimate K_{To} and μₛ, which are then used to update those parameters on the syringe control unit memory. FIG. 23 illustrates estimated versus measured pressure for the syringe assembly. FIG. 24 depicts transmission characterization at N=40. FIG. 25 depicts transmission characterization of a respective motor.

iii. Pressure Verification

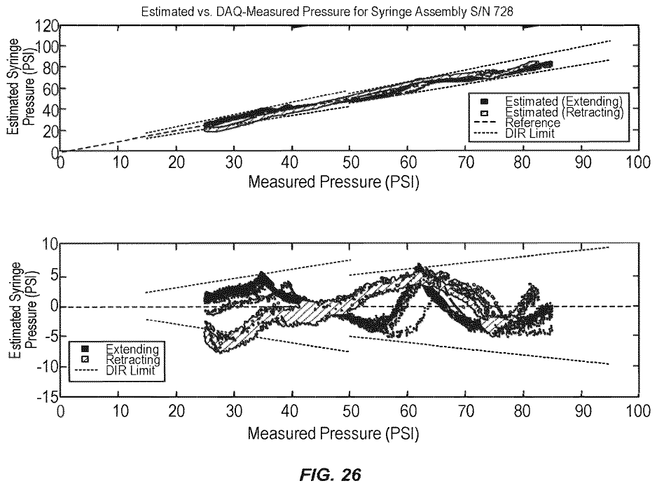

After calibration, the pressure can be verified by performing the pressure calibration with the updated K_{To} and μₛ, which is called pressure calibration verification. The estimated force data from the syringe can be plotted versus the measured force data, as shown in FIG. 26.

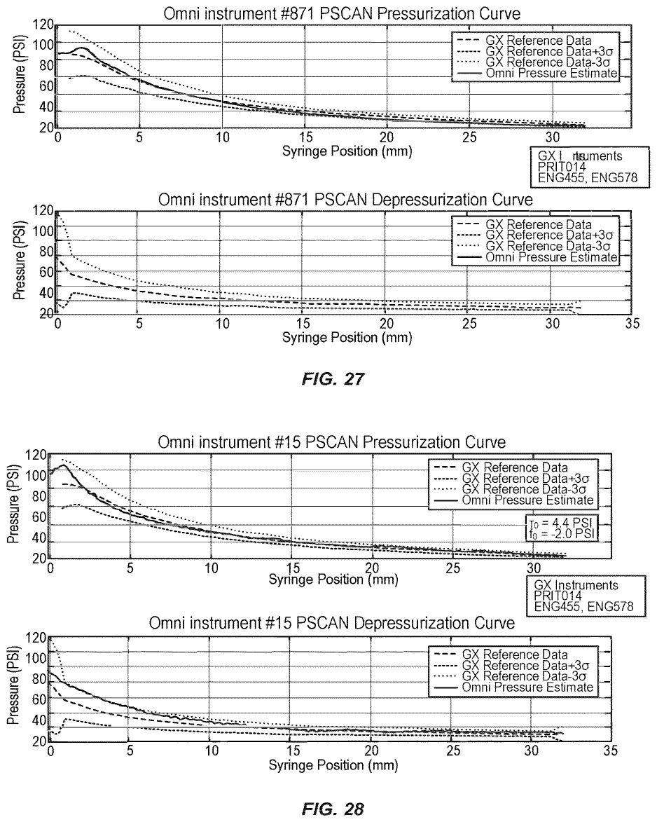

FIG. 27 shows a pressure comparison by using friction compensation methods during pressurization and depressurization to a first motor design. FIG. 28 shows a pressure comparison by using friction compensation methods during pressurization and depressurization by a second motor design.

iv. Cartridge Integrity Test

In another aspect, methods for cartridge integrity testing are provided herein. The cartridge integrity test (CIT) determines if there is leak in the reaction tube. This can be done by checking pressure differential between P1 and P2. P1 being the pressure at the end of first move against an open port (i.e., air chamber) and P2 being the pressure at the end of second move against reaction-vessel. Typically, P2-P1 should be greater than 4 PSI to pass the CIT. In some embodiments, the module can be configured to perform the CIT by introducing a delay at the end of second move (P2) to allow any slow leakage. This time delay can be configurable from CIT command. In the improved pressure sensing approaching described herein, the module may add the time delay in the CIT command to the P1 and P2 motion times.

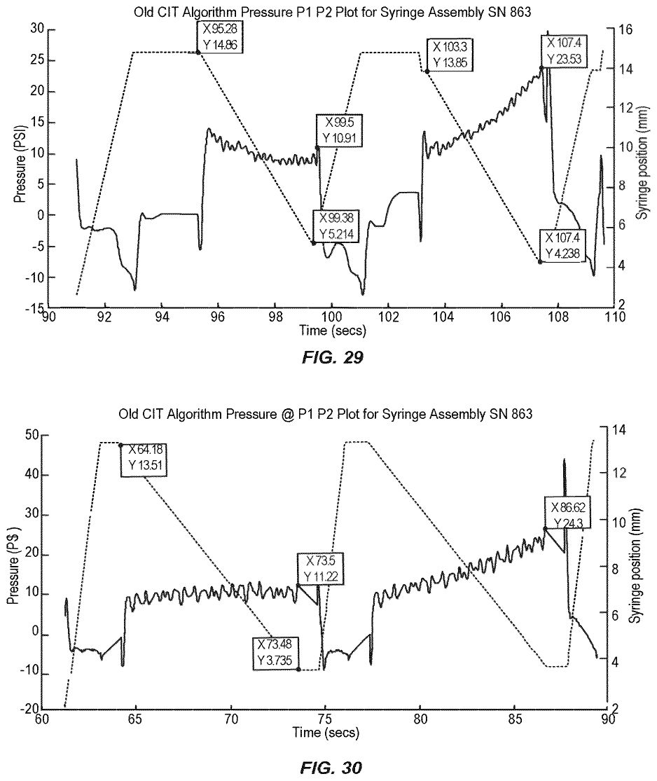

In the conventional module, a CIT algorithm includes a syringe dispense move for P2 starting at a lower position than a P1 dispense move, the velocity of moves is 200 μsteps/sec, and the time to complete move is 4 secs. The P1 and P2 moves are compound and have different start and end position such that P1 and P2 pressure are maximum values during move. Such a conventional CIT is shown in FIG. 29.

In utilizing the improved pressure sensing approach described herein, the CIT algorithm can include syringe dispense moves for P1 and P2 that start at same position. P1 and P2 dispense moves can be slower to allow the pressure to drop if there is a tiny leak in the reaction-vessel. Time to complete move is 4 secs+CIT time delay. P1 and P2 moves can be similar to conventional tests, but with P1 and P2 pressure measured at end of slew. An example of such a CIT is shown in FIG. 30.

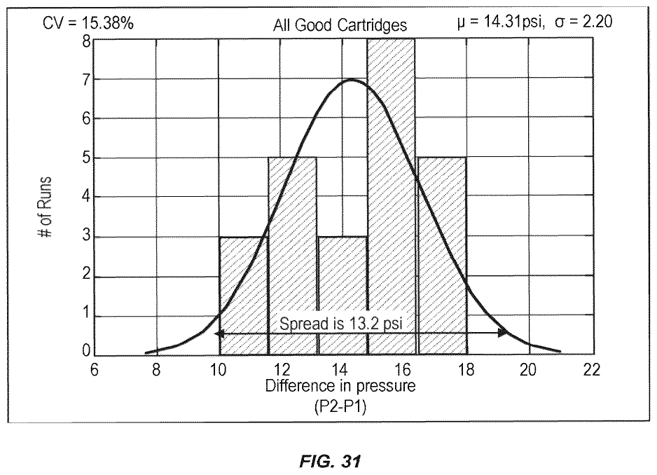

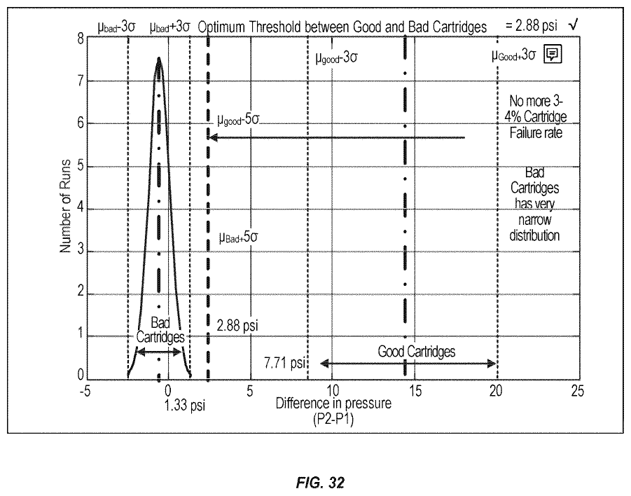

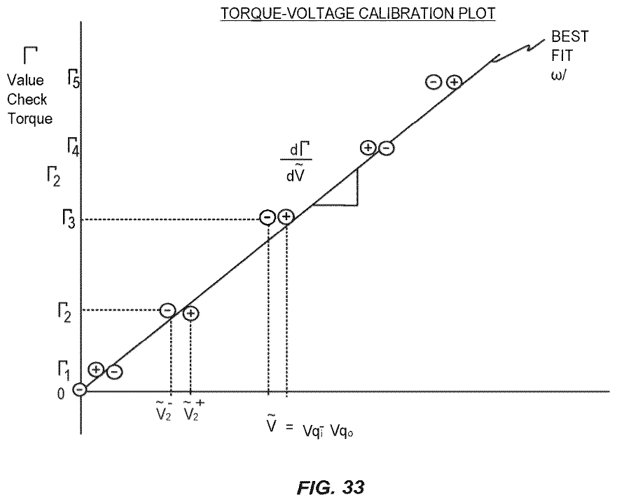

FIG. 31 illustrates CIT results from 9 modules, 2 types of cartridges and three different cartridge types. A total of 48 testing runs were conducted with 24 good cartridges and 24 bad cartridges (i.e. punctured cartridges). Each module is programed with software recorded on a memory of a processor module operably coupled thereto. FIG. 32 illustrates the optimum threshold to detect “good” versus “bad” cartridges.

v. Self Testing

In another aspect, the control unit can be configured to perform self-testing. A self-testing procedure tests the function of the system prior to running an assay. In some embodiments, a self-test procedure can be a relatively simple test that serves to demonstrate necessary and sufficient operating conditions to start an assay.

vi. Pressure Sensing Algorithm

In another aspect, an exemplary pressure sensing algorithm in accordance with the concepts described herein is provided as follows:

| Step | Description | Formula | Unit |
|---|---|---|---|
| 1 | Compute the applied voltage, V_{applied}. | ${V}_{applied} = {Voltage Feeback}^{} - {Motor Driver Crossover Distortion}^{} - {Motor Back - EMF}^{}$ | V |
| | across the motor | | |
| | windings. | | |
| 2 | Compute the resolved | i_{q} = V_{applied}/rₘ | A |
| | winding current, i_{q}. | | |
| 3 | Compute the motor torque, τₘ. | τₘ = (3/2)kₜ_{₀}i_{q} | N-m |
| 4 | Compute the net motor torque, τₙₑₜ, on the | ${net torque}^{} = {motor torque}^{} - {drag torque}^{}$ | N-m |
| | Syringe lead-screw nut. | | |
| 5 | Compute the theoretical force, \hat{f}ₙᵤₜ, on the | ${force transmitted to nut}^{} = {transmission ratio}^{} \times {net transmission torque}^{}$ | N |
| | Syringe nut. | | |
| 6 | Estimate the applied force on the syringe, f_{syringe} by applying | ${Syringe force estimate}^{} = \frac{\begin{matrix}theoretical force \\ transmitted by nut\end{matrix}}{friction scaling effect} = \frac{{f}_{nut}}{1 + sign ( {τ}_{m} {w}_{m} ) {μ}_{k} β}$ | N |
| | a syringe nut friction | | |
| | scaling factor. | | |
| 7 | Estimate the syringe pressure, \hat{p}_{syringe} by scaling the | ${\hat{p}}_{syringe} = \frac{{\hat{f}}_{syringe}}{{A}_{syringe cross - sectional area}}$ | PSI |
| | estimated force by | | |
| | the syringe bore | | |
| | cross-sectional area. | | |

The details for each of the above steps are described further below.

| Step | Details |
|---|---|
| 1 | Here, the applied motor winding voltage comprises the voltage feedback from the PID compensator in the |
| | motor position loop; the motor driver |
| 2 | Here we compute the motor current as resolved into a stationary coordinate system (d, q). The stationary |
| | coordinate system allows us to treat the three-phase brushless-motor as a single- phase, DC motor. i_{q} is |
| | torque-producing current in the motor. rₘ is the winding-to-neutral motor resistance. |
| 3 | For a brushless motor the motor torque is the average torque produced by each winding over a motor |
| | revolution times the number of phases, Nφ. For the Omni motors, Nφ is three—there are three phases in |
| | each of the brushless motors, so, |
| | τₘ = Nφτ̅_{winding} = (3/2)kₜ_{₀}i_{q} |
| 4 | There is a drag torque, τ₀, that acts in opposition to the direction of motion. Here we subtract this to |
| | obtain the torque, net of friction, on the screw. |
| 5 | The force generated by the transmission is the transmission ratio which is the input displacement divided |
| | by the corresponding to the output displacement. For a lead-screw of pitch, p, the transmission ratio is |
| | $\frac{motor rotation input}{nutdisplacement} = \frac{2 π}{p} \frac{radians}{meter}$ |
| 6 | Here, we apply the multiplicative friction scaling effect |
| | $\frac{1}{1 + sign ( {r}_{m} {w}_{m} ) {μ}_{k} β}$ |
| | onto the theoretical force to determine the syringe force. In this, μₖ is the kinetic coefficient of friction |
| | of the screw and b is a known transmission constant equal to the transmission ratio times the equivalent |
| | radius of the lead-screw threads, req. For the Syringe transmission, b is approximately 1. If positive |
| | motor power (an extension for instance) is applied against a positive pressure (force), the theoretical force |
| | overestimates the syringe force because the transmission must overcome the nut friction. So the friction |
| | scaling effect attenuates the syringe force estimate. Conversely, if negative motor power (in retraction |
| | for instance), the theoretical force underestimates the syringe force because the transmission is aided by |
| | the friction as it withstands the force. So the friction scaling effect amplifies the syringe force estimate to |
| | compute the force in that case. |
| 7 | We simply scale the syringe force estimate by the syringe bore area to obtain the pressure estimate. |

The pressure sensing signal descriptions are found in Appendix A.

### B. Valve Torque Estimation

In another aspect, methods for valve torque estimation are provided. To calculate the value of K_{To}, a plot can be used, as shown in FIG. 37, which represents a best fit and assumes an intercept at 0. The slope estimate can then be used to determine the value of K_{To} from the following equation:

$$\frac{}{} = \frac{3}{2} ( \frac{{K}_{g} {K}_{{T}_{o}}}{{r}_{n}} )$$

From this equation, the torque constant estimate, K_{To}, can be computed as:

$$\hat{{K}_{{T}_{o}}} = ( \frac{2}{3} ) \frac{{r}_{μ}}{{h}_{g}} \frac{{d}_{\hat{v}}}{}$$

The methods, systems, and devices discussed above are examples and it is appreciated that variations of the algorithms and examples can be realized and still be in keeping with the inventive concepts described herein. Various configurations can omit, substitute, or add various procedures or components as appropriate. For instance, in alternative configurations, the methods can be performed in an order different from that described, and/or various stages can be added, omitted, and/or combined. Also, features described with respect to certain configurations can be combined in various other configurations. Different aspects and elements of the configurations can be combined in a similar manner. Also, technology evolves and some of the elements as described are provided as non-limiting examples and thus do not limit the scope of the disclosure or claims.

Specific details are given in the description to provide a thorough understanding of exemplary configurations (including implementations). However, configurations can be practiced without these specific details. For example, well-known circuits, processes, algorithms, structures, and techniques have been shown without unnecessary detail in order to avoid obscuring the configurations. This description provides exemplary configurations that do not limit the scope, applicability, or configurations of the claims. Rather, the preceding description of the configurations will provide those skilled in the art with an enabling description for implementing described techniques. Various changes can be made in the function and arrangement of elements without departing from the spirit or scope of the disclosure.

Also, configurations can be described as a process which is depicted as a flow diagram or block diagram. Although each can describe the operations as a sequential process, some of the operations can be performed in parallel or concurrently. Furthermore, examples of the methods can be implemented by hardware, software, firmware, middleware, microcode, hardware description languages, or any combination thereof. When implemented in software, firmware, middleware, or microcode, the program code or code segments to perform the necessary tasks can be stored in a non-transitory computer-readable medium such as a storage medium. Processors can perform the described tasks.

Having described several exemplary configurations, various modifications, alternative constructions, and equivalents can be used without departing from the spirit of the disclosure. For example, the above elements can be components of a larger system, wherein other rules can take precedence over or otherwise modify the application of the invention. Also, a number of steps can be undertaken before, during, or after the above elements are considered. Accordingly, the above description does not bound the scope of the claims. All patents, patent applications, and other publications cited in this application are incorporated by reference in their entirety for all purposes.

1. A lossy mechatronic system for controlling at least one of a position, velocity or generalized force, the system comprising:
a motor driver;
a motor configured to apply a generalized force in accordance with the motor driver;
a lossy transmission configured to deliver a generalized force in accordance with the motor-applied generalized force, friction, and viscous drag; and
a control unit having a processor with a memory having instructions recorded thereon to compute, in real-time, the generalized force using a motor drive bridge current, a voltage, and a transmission characteristic comprising at least a coefficient of friction.

2. The system of claim 1, wherein the control unit is configured to compute the generalized force further using a motor characteristic comprising any of:
a velocity, a position, a phase current, a phase resistance, and a motor constant (kt).

3. The system of claim 1, wherein the transmission characteristic further comprises a viscous drag coefficient.

4. The system of claim 1, wherein the transmission is backdrivable enabling four-quadrant operation.

5. The system of claim 4, wherein a user of the system can impart generalized forces on an output and sense the generalized force at an input, thereby communicate user intent.

6. The system of claim 5, wherein the system includes a cartridge loading system configured such that a user pushing on the cartridge signals a user request to load the cartridge and start processing the cartridge.

7. The system of claim 1, wherein the transmission is a rotary transmission with an output torque representing the generalized force output.

8. The system of claim 1, wherein the transmission is a linear transmission with an output force representing the generalized force output.

9. The system of claim 1, wherein the system is applied in at least one of: a syringe, a valve, a cartridge loading mechanism, and a door opening/closing mechanism.

10. The system of claim 1, wherein the control unit is configured to:
determine a motor resistance by a motor drive voltage, a motor drive bridge current and a motor drive bridge voltage.

11. The system of claim 10, where the motor comprises motor windings are of known conductor composition, where the motor resistance is further determined at a known winding temperature, which are stored in the memory of the control unit and also in real-time, the motor winding temperature determined from a known relationship between motor winding resistance and the winding temperature.

12. The system of claim 11, wherein the motor windings are constructed with substantially copper composition.

13. The system of claim 12, where the motor winding temperature is used to compensate for an impact of winding temperature on the generalized force output.

14. The system of claim 12, where operation of the system is shut down when the motor winding temperature exceeds a pre-determined threshold.

15. The system of claim 1, wherein the system includes a syringe and the generalized force output is used in a guarded, stop-on-force motion of the syringe during at least one of the following operations:
locating a cartridge bottom with the syringe,
detecting excessive aspirating or dispensing force while performing at least one of mixing or reaction-tube filling with the syringe, and
determining a sample-volume adequacy.

16. The system of claim 15, wherein the guarded, stop-on-force motion is a stop-on-pressure.

17. The system of claim 1, wherein the system is applied as a syringe and the control unit is configured such that the generalized force output is used during a cartridge integrity test to determine a cartridge integrity.

18. The system of claim 17, wherein the cartridge integrity is determined by sensing a loss of pressurization due to a leak in a reaction-vessel.

19. A calibration method for application to a lossy mechatronic system, the calibration method comprising:
obtaining at least one of an assumed nominal winding resistance or a determined motor winding resistance, for a motor of the lossy mechatronic system;
extending a transmission of the lossy mechatronic system and then retracting the transmission while driving into a compliant, instrumented platform;
recording a reading from the instrumented platform and a generalized force; and
computing, by processing the recordings by the platform, a coefficient-of-friction, wherein a control unit, having a processor with a memory having instructions recorded thereon, computes, in real-time, at least one other generalized force, deliverable by the transmission, by a computation using a motor drive bridge current, a voltage, and a transmission characteristic that includes at least the coefficient-of-friction.

20. The method of claim 19, wherein the system output is linear.

21. The method of claim 20, wherein the linear output system is a syringe.

22. The method of claim 19, wherein the coefficient-of-friction is stored on a memory of a control unit of the lossy mechatronic system to facilitate accurate operation of the lossy mechatronic system within a +/−10% accuracy.

23. The method of claim 19, further comprising:
calibrating a motor constant (kt) and the coefficient of friction (μs) based on a pressure sensing calibration procedure performed for the lossy mechatronic system;
wherein the computation of the at least one other generalized force, deliverable by the transmission, uses a motor characteristic that includes at least the calibrated motor constant (kt), the motor drive bridge current, the voltage, and the transmission characteristic that includes the calibrated coefficient of friction (μs).

24. The method of claim 23, wherein the motor characteristic further comprises one or more of:
a velocity, a position, a phase current, and a phase resistance.

25. The system of claim 1, wherein the control unit to compute, in real-time, the generalized force is configured to compute the generalized force further using a calibrated motor constant (kt), wherein the coefficient-of-friction (μs) is a calibrated coefficient of friction, and wherein the calibrated motor constant (kt) and the calibrated coefficient-of-friction (μs) are determined based on a pressure sensing calibration procedure performed for the lossy mechatronic system.
